(19) United States
(12) Patent Application Publication
(10) Pub. No.: US 2020/0138327 A1
(43) Pub. Date: May 7, 2020

(54) BIOMETRIC ANTENNA DEVICE, PULSE WAVE MEASUREMENT DEVICE, BLOOD PRESSURE MEASUREMENT DEVICE, APPARATUS, BIOLOGICAL INFORMATION MEASUREMENT METHOD, PULSE WAVE MEASUREMENT METHOD, AND BLOOD PRESSURE MEASUREMENT METHOD

(71) Applicants: OMRON Corporation, Kyoto (JP); OMRON HEALTHCARE Co., Ltd., Kyoto (JP)

(72) Inventors: Hisashi Ozawa, Kyoto-shi (JP); Keisuke Saito, Osaka (JP); Keigo Kamada, Tokyo (JP); Yasuhiro Kawabata, Kyoto (JP)

(73) Assignees: OMRON Corporation, Kyoto (JP); OMRON HEALTHCARE Co., Ltd., Kyoto (JP)

(21) Appl. No.: 16/736,515

(22) Filed: Jan. 7, 2020

Related U.S. Application Data
(63) Continuation of application No. PCT/JP2018/024043, filed on Jun. 25, 2018.

(30) Foreign Application Priority Data
Jul. 21, 2017 (JP) 2017-142231

(51) Int. Cl. A61B 5/05; A61B 5/021; A61B 5/024; A61B 5/00; G01S 13/88

(52) U.S. Cl. CPC A61B 5/05; A61B 5/02125; G01S 13/88; A61B 5/6831; A61B 5/02444

(57) ABSTRACT

A biometric antenna device of the present invention includes: a conductor layer configured to face the measurement site for emitting and/or receiving the radio wave; and a dielectric layer mounted along a facing surface facing the measurement site of the conductor layer or of a base material mounting the conductor layer and extending in parallel with the conductor layer, the dielectric layer having a predetermined relative permittivity. The dielectric layer keeps a distance between an outer surface of the measurement site and the conductor layer constant, in a mounted state in which a second surface on a side opposite to a side of a first surface on a side along the conductor layer of the dielectric layer abuts on an outer surface of the measurement site.

### CROSS-REFERENCE TO RELATED APPLICATION

This is a continuation application of International Application No. PCT/JP2018/024043, with an International filing date of Jun. 25, 2018, which claims priority of Japanese Patent Application No. 2017-142231 filed on Jul. 21, 2017, the entire content of which is hereby incorporated by reference.

### TECHNICAL FIELD

The present invention relates to a biometric antenna device, and more particularly to a biometric antenna device that emits radio waves toward a measurement site of a living body or receives radio waves from the measurement site for measurement of biological information. In addition, the present invention relates to a pulse wave measurement device, a blood pressure measurement device, and an apparatus including the biometric antenna device. In addition, the present invention relates to a biological information measurement method for emitting radio waves toward a measurement site of a living body or receiving radio waves from the measurement site. In addition, the present invention relates to a pulse wave measurement method and a blood pressure measurement method including the biological information measurement method.

### BACKGROUND ART

Conventionally, as this kind of biometric antenna device, for example, as disclosed in Patent Document 1 (Japanese Patent No. 5879407), a biometric antenna device is known that includes a transmitting (emitting) antenna and a receiving antenna facing a measurement site, that emits a radio wave (measurement signal) from the transmitting antenna toward the measurement site (target object), that receives a radio wave reflected by this measurement site (reflected signal) by the receiving antenna, and that measures biological information.

### SUMMARY OF THE INVENTION

However, in Patent Document 1, there is no disclosure or suggestion how to arrange the transmitting antenna and the receiving antenna (appropriately, these are collectively referred to as “transmitting and receiving antenna pair”) at a predetermined distance with respect to the measurement site. For example, when the measurement site is the wrist, if the distance between the outer surface of the wrist and the transmitting and receiving antenna pair varies with each measurement, the received signal level varies, and there arises a problem that biological information cannot be measured with high precision.

Thus, an object of the present invention is to provide a biometric antenna device capable of keeping a conductor layer forming a transmitting and receiving antenna pair at a predetermined distance with respect to a measurement site, and therefore capable of measuring biological information from the measurement site with high precision. In addition, an object of the present invention is to provide a pulse wave measurement device, a blood pressure measurement device, and an apparatus including the biometric antenna device. In addition, an object of the present invention is to provide a biological information measurement method for measuring biological information from a measurement site using the biometric antenna device. In addition, the present invention is to provide a pulse wave measurement method and a blood pressure measurement method including the biological information measurement method.

In order to solve the above-mentioned problem, a biometric antenna device of the present invention for emitting a radio wave toward a measurement site of a living body or for receiving a radio wave from the measurement site, the biometric antenna device comprises:

a conductor layer configured to face the measurement site for emitting and/or receiving the radio wave; and

a dielectric layer mounted along a facing surface facing the measurement site of the conductor layer or of a base material mounting the conductor layer and extending in parallel with the conductor layer, the dielectric layer having a predetermined relative permittivity,

wherein the dielectric layer keeps a distance between an outer surface of the measurement site and the conductor layer constant, in a mounted state in which a second surface on a side opposite to a side of a first surface on a side along the conductor layer of the dielectric layer abuts on an outer surface of the measurement site.

In the present specification, the “measurement site” may be a trunk in addition to a rod-shaped site such as an upper limb (wrist, upper arm, or the like) or a lower limb (ankle, or the like).

In addition, the “outer surface” of the measurement site refers to a surface exposed to the outside. For example, if the measurement site is a wrist, it refers to the outer peripheral surface of the wrist or a part thereof (for example, the palmar surface corresponding to the palmar side portion in the circumferential direction of the outer peripheral surface).

In addition, the “conductor layer” can be used, for emitting and/or receiving a radio wave, as a transmitting antenna or a receiving antenna, or as a transmitting and receiving shared antenna via a known circulator. The “conductor layer” may be divided into a transmitting antenna and a receiving antenna that receives a radio wave from the transmitting antenna.

In addition, unless otherwise noted, the “predetermined relative permittivity” of the dielectric layer may be uniform over the range in which the dielectric layer occupies space, or may vary depending on the position within a range in which the dielectric layer occupies space.

In addition, a phrase that the dielectric layer “keeps a distance constant” between an outer surface of the measurement site and the conductor layer means that the dielectric layer is a spacer. It should be noted that in the case where the dielectric layer has flexibility, it means acceptable that the “distance” more or less fluctuates due to the bending when it is bent by an external force.

In another aspect, a pulse wave measurement device of the present disclosure for measuring a pulse wave of a measurement site of a living body, the pulse wave measurement device comprises:

the biometric antenna device;

wherein the second surface of the dielectric layer is configured to abut on an outer surface of the measurement site, and a transmitting and receiving antenna pair including a transmitting antenna and a receiving antenna formed by the conductor layer is configured to correspond to an artery passing through the measurement site in a mounted state in which the belt is mounted to wind around an outer surface of the measurement site,

a transmitting circuit configured to emit a radio wave toward the measurement site via the transmitting antenna;

a receiving circuit configured to receive a radio wave reflected by the measurement site via the receiving antenna; and

a pulse wave detection unit configured to acquire a pulse wave signal representing a pulse wave of an artery passing through the measurement site based on an output of the receiving circuit.

Here, when the conductor layer is divided into a transmitting antenna and a receiving antenna that receives a radio wave from the transmitting antenna in the surface direction perpendicular to the thickness direction of the conductor layer, the “transmitting and receiving antenna pair” refers to the transmitting antenna and the receiving antenna. In addition, when the conductor layer spatially forms one transmitting and receiving shared antenna, all of the “transmitting antenna”, the “receiving antenna”, and the “transmitting and receiving antenna pair” refer to the transmitting and receiving shared antenna.

In another aspect, a blood pressure measurement device of the present disclosure for measuring blood pressure of a measurement site of a living body, the blood pressure measurement device comprises:

the two sets of pulse wave measurement devices;

wherein a belt in the two sets is integrally formed,

wherein transmitting and receiving antenna pairs in the two sets are arranged apart from each other in a width direction of the belt,

wherein in a mounted state where the belt is mounted to wind around an outer surface of the measurement site, the second surface of the dielectric layer abuts on an outer surface of the measurement site, and a first set of transmitting and receiving antenna pair of the two sets corresponds to an upstream side portion of an artery passing through the measurement site, while a second set of transmitting and receiving antenna pair corresponds to a downstream side portion of the artery,

wherein in each of the two sets, the transmitting circuit emits a radio wave toward the measurement site via the transmitting antenna, and the receiving circuit receives a radio wave reflected by the measurement site via the receiving antenna, and

wherein in each of the two sets, the pulse wave detection unit acquires a pulse wave signal representing a pulse wave of an artery passing through the measurement site based on an output of the receiving circuit,

a time difference acquisition unit configured to acquire a time difference between pulse wave signals acquired by the two sets of respective pulse wave detection units as a pulse transit time; and

a first blood pressure calculation unit configured to calculate a blood pressure value based on a pulse transit time acquired by the time difference acquisition unit by using a predetermined correspondence formula between a pulse transit time and a blood pressure.

In another aspect, an apparatus of the present disclosure comprises:

the biometric antenna device;

the pulse wave measurement device; or

the blood pressure measurement device.

In another aspect, a biological information measurement method of the present disclosure for acquiring biological information from a measurement site of a living body by using the biometric antenna device, the biological information measurement method comprises:

causing the second surface of the dielectric layer to abut on an outer surface of the measurement site to mount the biometric antenna device on the measurement site; and

in a mounted state where the dielectric layer keeps a distance between an outer surface of the measurement site and the conductor layer constant, emitting a radio wave from the conductor layer toward the measurement site through the dielectric layer or a gap present on a side of the dielectric layer, and/or receiving a radio wave reflected by the measurement site with the conductor layer through the dielectric layer or a gap present on a side of the dielectric layer.

In another aspect, a pulse wave measurement method of the present disclosure for measuring a pulse wave of a measurement site of a living body by using the pulse wave measurement device, the pulse wave measurement method comprises:

mounting the belt to wind around an outer surface of the measurement site, causing the second surface of the dielectric layer to abut on an outer surface of the measurement site, and causing a transmitting and receiving antenna pair including a transmitting antenna and a receiving antenna formed by the conductor layer to correspond to an artery passing through the measurement site;

in a mounted state in which the dielectric layer keeps a distance between the measurement site and the conductor layer constant, emitting a radio wave toward the measurement site with the transmitting circuit via the transmitting antenna, and receiving a radio wave reflected by the measurement site with the receiving circuit via the receiving antenna; and

acquiring a pulse wave signal representing a pulse wave of an artery passing through the measurement site with the pulse wave detecting unit based on an output of the receiving circuit.

In another aspect, a blood pressure measurement method of the present disclosure for measuring blood pressure of a measurement site of a living body by using the blood pressure measurement device, the blood pressure measurement method comprises:

mounting the belt to wind around an outer surface of the measurement site, causing the second surface of the dielectric layer to abut on an outer surface of the measurement site, and causing a first set of transmitting and receiving antenna pair of the two sets to correspond to an upstream side portion of an artery passing through the measurement site, while causing a second set of transmitting and receiving antenna pair to correspond to a downstream side portion of the artery;

in a mounted state where the dielectric layer keeps a distance between the measurement site and the conductor layer constant, in each of the two sets, emitting a radio wave toward the measurement site with the transmitting circuit via the transmitting antenna, and receiving a radio wave reflected by the measurement site with the receiving circuit via the receiving antenna;

in each of the two sets, acquiring a pulse wave signal representing a pulse wave of an artery passing through the measurement site with the pulse wave detection unit based on an output of the receiving circuit;

acquiring a time difference between pulse wave signals acquired by the two sets of respective pulse wave detection units with the time difference acquisition unit as a pulse transit time; and

calculating a blood pressure value with the first blood pressure calculation unit based on a pulse transit time acquired by the time difference acquisition unit by using a predetermined correspondence formula between a pulse transit time and a blood pressure.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view illustrating an appearance of a wrist sphygmomanometer of an embodiment according to a biometric antenna device, a pulse wave measurement device, and a blood pressure measurement device of the present invention.

FIG. 2 is a diagram schematically illustrating a cross section perpendicular to the longitudinal direction of the wrist in a state where the sphygmomanometer is mounted on the left wrist.

FIG. 3 is a diagram illustrating a planar layout of a transmitting and receiving antenna group constituting first and second pulse wave sensors in a state where the sphygmomanometer is mounted on the left wrist.

FIG. 4 is a diagram illustrating an overall block configuration of a control system of the sphygmomanometer.

FIG. 5 is a diagram illustrating a partial and functional block configuration of a control system of the sphygmomanometer.

FIG. 6 is a diagram illustrating a cross-sectional structure of an example of a transmitting antenna or a receiving antenna included in the transmitting and receiving antenna group in a state of being mounted on the left wrist.

FIG. 7 is a diagram illustrating a cross-sectional structure of another example of a transmitting antenna or a receiving antenna in a state of being mounted on the left wrist.

FIG. 8A is a diagram schematically illustrating a cross section along the longitudinal direction of the wrist in a state where the sphygmomanometer is mounted on the left wrist. FIG. 8B is a diagram illustrating waveforms of first and second pulse wave signals output from the first and second pulse wave sensors, respectively.

FIG. 9A is a diagram illustrating a block configuration implemented by a program for performing an oscillometric method in the sphygmomanometer.

FIG. 9B is a diagram illustrating an operation flow when the sphygmomanometer performs blood pressure measurement by the oscillometric method.

FIG. 10 is a diagram illustrating changes in a cuff pressure and a pulse wave signal according to the operation flow in FIG. 9B.

FIG. 11 is a diagram illustrating an operation flow according to the biological information measurement method, pulse wave measurement method, and blood pressure measurement method of one embodiment of the present invention; the operation flow including: the sphygmomanometer performing pulse wave measurement, acquiring a pulse transit time (PTT), and performing blood pressure measurement (estimation) based on the pulse transit time.

FIG. 12 is a diagram schematically illustrating an example of a mode in which a belt is mounted on the left wrist together with a transmitting antenna or a receiving antenna in a cross section perpendicular to the longitudinal direction of the left wrist.

FIG. 13 is a diagram schematically illustrating another example of a mode in which a belt is mounted on the left wrist together with a transmitting antenna or a receiving antenna in a cross section perpendicular to the longitudinal direction of the left wrist.

FIG. 14 is a diagram schematically illustrating still another example of a mode in which a belt is mounted on the left wrist together with a transmitting antenna or a receiving antenna in a cross section perpendicular to the longitudinal direction of the left wrist.

FIG. 15 is a diagram schematically illustrating still another example of a mode in which a belt is mounted on the left wrist together with a transmitting antenna or a receiving antenna in a cross section perpendicular to the longitudinal direction of the left wrist.

FIG. 16 is a diagram schematically illustrating still another example of a mode in which a belt is mounted on the left wrist together with a transmitting antenna or a receiving antenna in a cross section perpendicular to the longitudinal direction of the left wrist.

FIG. 17 is a diagram illustrating another mode of the dielectric layer constituting the transmitting antenna or the receiving antenna.

FIGS. 18A and 18B are diagrams for illustrating the effect of the dielectric layer being interposed between the palmar surface of the left wrist and the conductor layer.

FIG. 19A is a diagram illustrating a cross-sectional structure of a modified example of a transmitting antenna or a receiving antenna in a state of being mounted on the left wrist. FIG. 19B is a diagram illustrating a transmitting and receiving antenna pair corresponding to FIG. 19A as viewed obliquely.

### DESCRIPTION OF EMBODIMENTS

Hereinafter, embodiments of the present invention will be described in detail with reference to the drawings.

(Configuration of Sphygmomanometer)

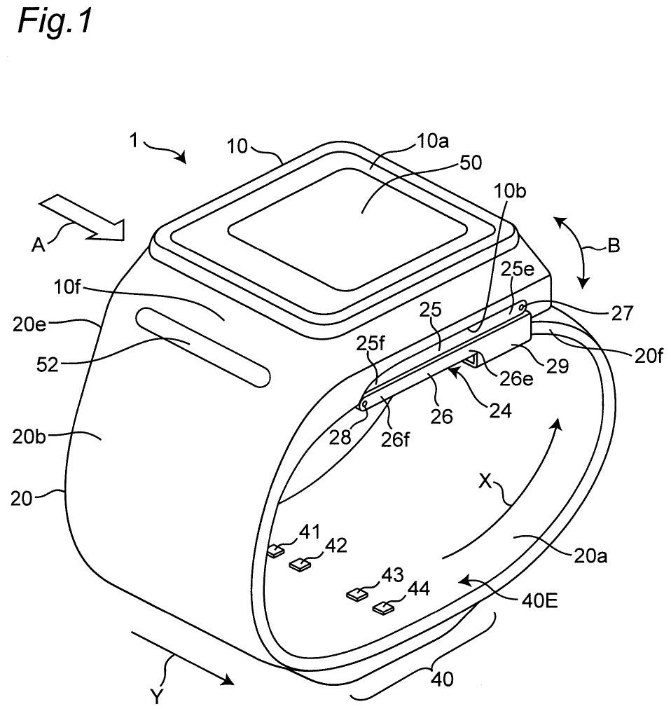

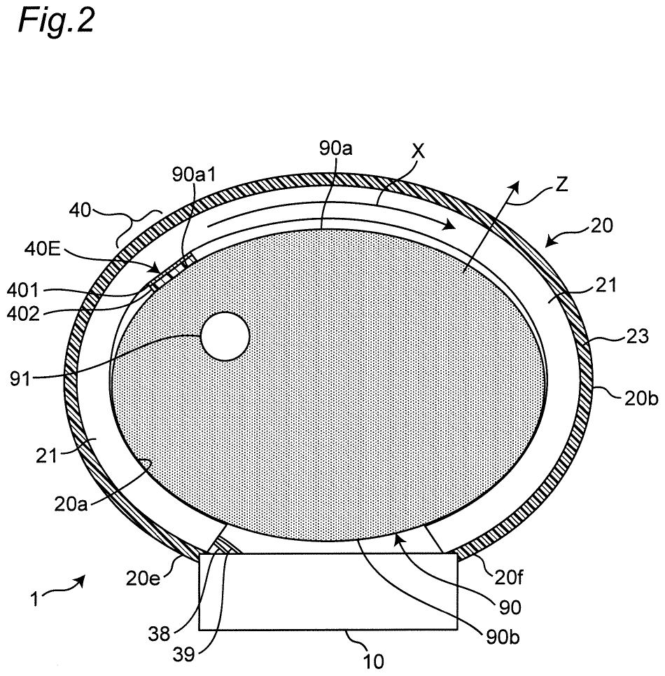

FIG. 1 illustrates the appearance of a wrist-type sphygmomanometer (the whole is denoted by reference numeral 1) of one embodiment according to a biometric antenna device, a pulse wave measurement device, and a blood pressure measurement device of the present invention as viewed from an oblique direction. In addition, FIG. 2 schematically illustrates a cross section perpendicular to the longitudinal direction of the left wrist 90 in a state where the sphygmomanometer 1 is mounted on the left wrist 90 as a measurement site (hereinafter referred to as “mounted state”).

As illustrated in these drawings, the sphygmomanometer 1 broadly includes a belt 20 to be mounted around a user's left wrist 90 and a main body 10 integrally attached to the belt 20.

As understood from FIG. 1, the belt 20 has an elongated belt shape to wind around the left wrist 90 along the circumferential direction, an inner peripheral surface 20a to be in contact with the left wrist 90, and an outer peripheral surface 20b opposite to the inner peripheral surface 20a. The dimension (width dimension) in the width direction Y of the belt 20 is set to about 30 mm in this example.

The main body 10 is integrally provided at one end portion 20e of the belt 20 in the circumferential direction by integral molding in this example. It should be noted that the belt 20 and the main body 10 may be separately formed, and the main body 10 may be integrally attached to the belt 20 via an engaging member (for example, a hinge or the like). In this example, the site where the main body 10 is disposed is intended to correspond to the back side surface of the left wrist 90 (the surface on the back side of the hand) 90b in the mounted state (see FIG. 2). In FIG. 2, a radial artery 91 passing near the palmar surface (surface on the palmar side) 90a as an outer surface in the left wrist 90 is illustrated.

As understood from FIG. 1, the main body 10 has a three-dimensional shape having a thickness in a direction perpendicular to the outer peripheral surface 20b of the belt 20. The main body 10 is formed small and thin so as not to interfere with the daily activities of the user. In this example, the main body 10 has a truncated quadrangular pyramid-shaped contour projecting outward from the belt 20.

A display 50 serving as a display screen is provided on the top surface 10a of the main body 10 (the surface on a side farthest from the measurement site). In addition, an operation unit 52 for inputting instructions from the user is provided along the side surface 10f of the main body 10 (side surface on the left front side in FIG. 1).

A transmission and reception unit 40 constituting first and second pulse wave sensors is provided in a site between one end portion 20e and the other end portion 20f in the circumferential direction of the belt 20. Of the belt 20, on the inner peripheral surface 20a of the site where the transmission and reception unit 40 is disposed, four transmitting and receiving antennas 41 to 44 (all of which are referred to as “transmitting and receiving antenna group” and denoted by reference numeral 40E) are mounted in a state of being separated from each other in the width direction Y of the belt 20 (described in detail below). In this example, the site where the transmitting and receiving antenna group 40E is disposed in the longitudinal direction X of the belt 20 is intended to correspond to the radial artery 91 of the left wrist 90 in the mounted state (see FIG. 2).

As illustrated in FIG. 1, the bottom surface 10b of the main body 10 (the surface on the side closest to the measurement site) and the end portion 20f of the belt 20 are connected by a threefold buckle 24. The buckle 24 includes a first plate-shaped member 25 disposed on the outer peripheral side and a second plate-shaped member 26 disposed on the inner peripheral side. One end portion 25e of the first plate-shaped member 25 is rotatably attached to the main body 10 via a coupling rod 27 extending along the width direction Y. The other end portion 25f of the first plate-shaped member 25 is rotatably attached to one end portion 26e of the second plate-shaped member 26 via a coupling rod 28 extending along the width direction Y. The other end portion 26f of the second plate-shaped member 26 is fixed near the end portion 20f of the belt 20 by the fixing portion 29. It should be noted that the attaching position of the fixing portion 29 in the longitudinal direction X of the belt 20 (corresponding to the circumferential direction of the left wrist 90 in the mounted state) is variably set in advance in accordance with the circumferential length of the left wrist 90 of the user. Thus, the sphygmomanometer 1 (belt 20) is formed in a substantially annular shape as a whole, and the bottom surface 10b of the main body 10 and the end portion 20f of the belt 20 can be opened and closed in the arrow B direction by the buckle 24.

When mounting the sphygmomanometer 1 on the left wrist 90, the user inserts the left hand into the belt 20 in the direction indicated by the arrow A in FIG. 1 with the buckle 24 open and the diameter of the ring of the belt 20 increased. Then, as illustrated in FIG. 2, the user adjusts the angular position of the belt 20 around the left wrist 90 to position the transmission and reception unit 40 of the belt 20 on the radial artery 91 passing through the left wrist 90. Thus, the transmitting and receiving antenna group 40E of the transmission and reception unit 40 abuts on a portion 90a1 corresponding to the radial artery 91 on the palmar surface 90a of the left wrist 90. In this state, the user closes and fixes the buckle 24. Thus, the user wears the sphygmomanometer 1 (belt 20) on the left wrist 90.

As illustrated in FIG. 2, in this example, the belt 20 includes a strip 23 forming the outer peripheral surface 20b and a pressing cuff 21 as a pressing member attached along the inner peripheral surface of the strip 23. The strip 23 is made of a plastic material (silicone resin in this example), and in this example, the strip 23 has flexibility in the thickness direction Z, and hardly stretches (substantially non-stretchable) in the longitudinal direction X (corresponding to the circumferential direction of the left wrist 90). In this example, the pressing cuff 21 is configured as a fluid bag by facing two stretchable polyurethane sheets in the thickness direction Z and welding their peripheral portions. The transmitting and receiving antenna group 40E of the transmission and reception unit 40 is disposed at a site corresponding to the radial artery 91 of the left wrist 90 on the inner peripheral surface 20a of the pressing cuff 21 (belt 20), as described above.

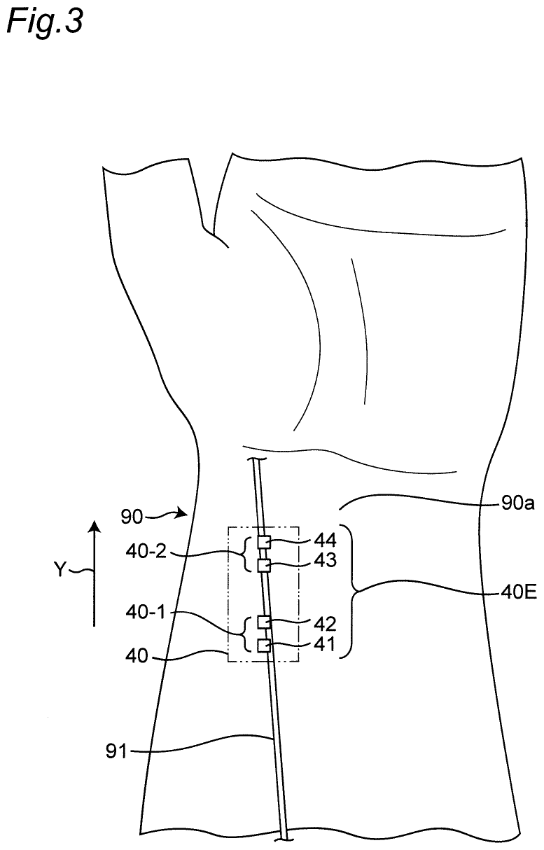

In this example, as illustrated in FIG. 3, in the mounted state, the transmitting and receiving antenna group 40E of the transmission and reception unit 40 is aligned separated from each other substantially along the longitudinal direction of the left wrist 90 (corresponding to the width direction Y of the belt 20) according to the radial artery 91 of the left wrist 90. In this example, in the width direction Y, the transmitting and receiving antenna group 40E includes transmitting antennas 41 and 44 disposed on both sides within the range occupied by the transmitting and receiving antenna group 40E, and receiving antennas 42 and 43 disposed between these transmitting antennas 41 and 44. The transmitting antenna 41 and the receiving antenna 42 for receiving a radio wave from the transmitting antenna 41 constitute a first set of transmitting and receiving antenna pair (41, 42) (The pair is shown in parentheses. The same applies hereinafter.). In addition, the transmitting antenna 44 and the receiving antenna 43 for receiving a radio wave from the transmitting antenna 44 constitute a second set of transmitting and receiving antenna pair (44, 43). In this arrangement, the transmitting antenna 41 is closer to the receiving antenna 42 than the transmitting antenna 44. In addition, the transmitting antenna 44 is closer to the receiving antenna 43 than the transmitting antenna 41. Therefore, interference between the first set of transmitting and receiving antenna pair (41, 42) and the second set of transmitting and receiving antenna pair (44, 43) can be reduced.

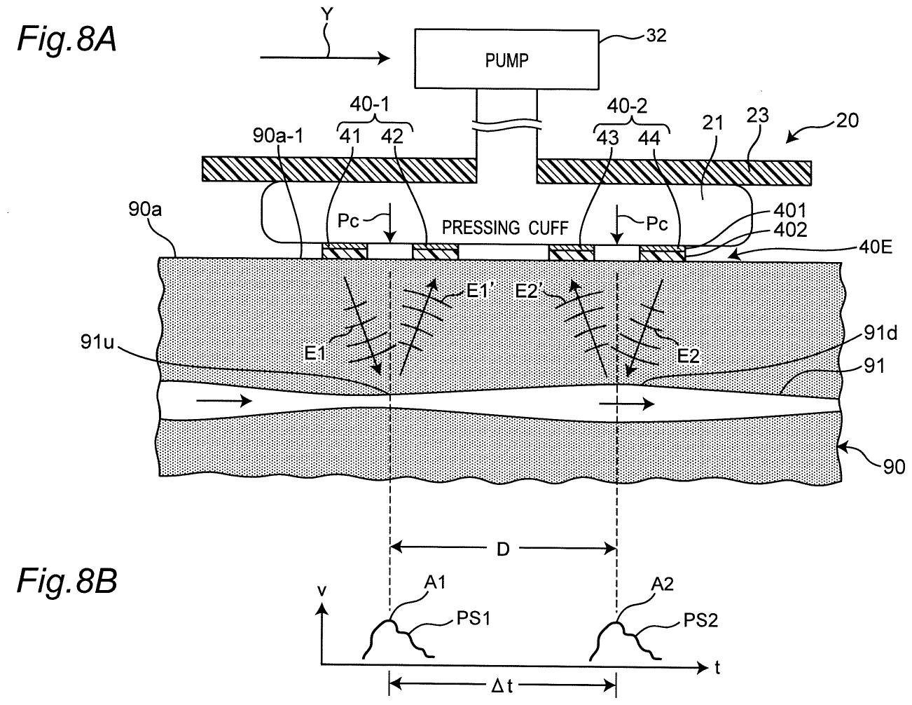

In this example, one transmitting antenna or receiving antenna has a square shape of 3 mm both in length and width (this shape in the surface direction is referred to as a “pattern shape”) in the surface direction (meaning the direction along the outer peripheral surface of the left wrist 90 in FIG. 3) so as to be able to emit or receive a radio wave at a frequency of 24 GHz band. In this example, in the width direction Y of the belt 20, the distance between the center of the transmitting antenna 41 and the center of the receiving antenna 42 in the first set is set within a range of 8 mm to 10 mm. Similarly, in this example, in the width direction Y of the belt 20, the distance between the center of the transmitting antenna 44 and the center of the receiving antenna 43 in the second set is set within a range of 8 mm to 10 mm. In addition, in the width direction Y of the belt 20, a distance D between the center of the first set of transmitting and receiving antenna pair (41, 42) and the center of the second set of transmitting and receiving antenna pair (44, 43) (see FIG. 8A) is set to 20 mm in this example. This distance D corresponds to a substantial space between the first set of transmitting and receiving antenna pair (41, 42) and the second set of transmitting and receiving antenna pair (44, 43). It should be noted that the length of the distance D or the like is an example, and an optimal length has only to be selected as appropriate according to the size or the like of the sphygmomanometer.

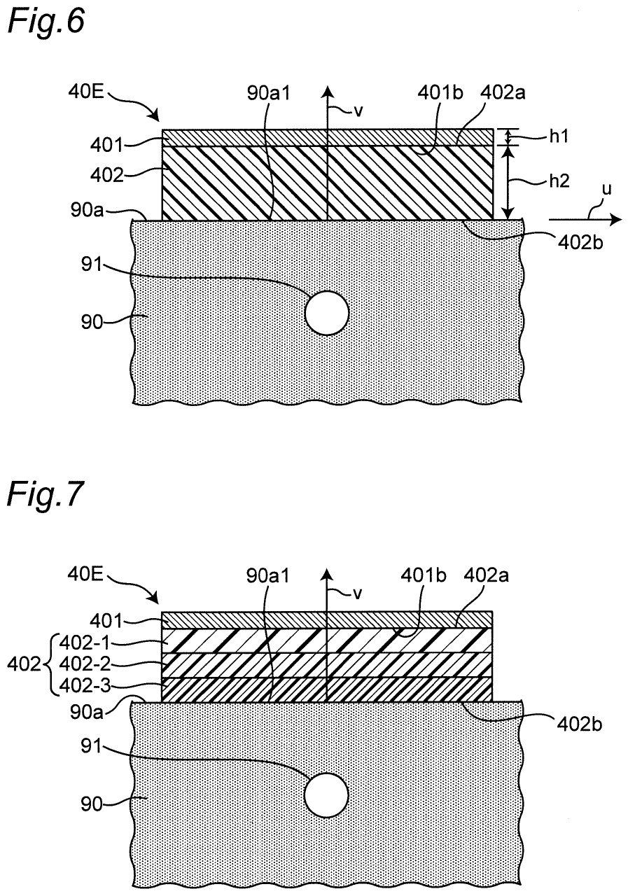

In addition, as shown in FIG. 6, in this example, the transmitting and receiving antenna group 40E includes a conductor layer 401 for emitting or receiving a radio wave. A dielectric layer 402 is attached along a facing surface 401b of the conductor layer 401 facing the left wrist 90 (the same configuration is used for each transmitting antenna and receiving antenna). The stacked structure of the conductor layer 401 and the dielectric layer 402 constitutes a biometric antenna device. In this example, although the pattern shape of the dielectric layer 402 is set to be the same as the pattern shape of the conductor layer 401, the pattern shapes may be different. In the mounted state where the transmitting and receiving antenna group 40E is mounted on the left wrist 90, a second surface 402b of the dielectric layer 402 on the side opposite to that of the first surface 402a on the side along the conductor layer 401 abuts on the palmar surface 90a of the left wrist 90. In this mounted state, the conductor layer 401 faces the palmar surface 90a of the left wrist 90, the dielectric layer 402 acts as a spacer, and the distance (distance in the thickness direction v) between the palmar surface 90a of the left wrist 90 and the conductor layer 401 (facing surface 401b) is kept constant.

In this example, the conductor layer 401 is made of metal (for example, copper). In this example, the dielectric layer 402 is made of polycarbonate, so that the relative permittivity of the dielectric layer 402 is uniformly set to εᵣ≈3.0. It should be noted that the relative permittivity means a relative permittivity at a frequency of 24 GHz band of radio waves used for transmission and reception (the same applies hereinafter).

This transmitting and receiving antenna group 40E can be configured to be flat along the surface direction u along the outer peripheral surface of the left wrist 90. Therefore, in the sphygmomanometer 1, the belt 20 can be configured to be thin as a whole. In this example, the thickness of the conductor layer 401 is set to h1=30 μm, and the thickness of the dielectric layer 402 is set to h2=2 mm.

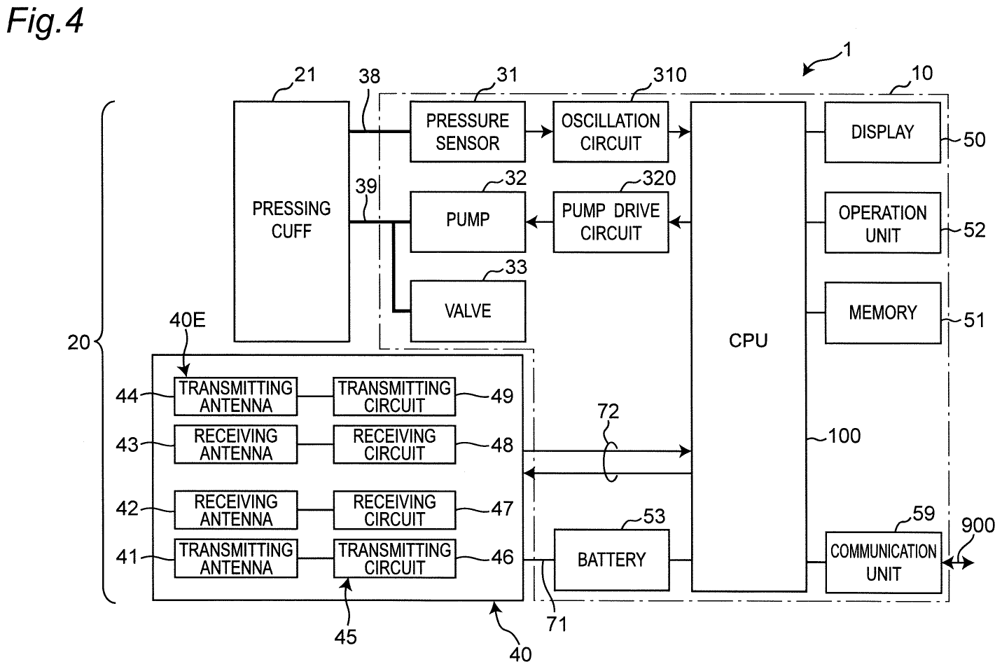

FIG. 4 illustrates an overall block configuration of a control system of the sphygmomanometer 1. In addition to the display 50 and the operation unit 52 described above, the main body 10 of the sphygmomanometer 1 mounts a central processing unit (CPU) 100 as a control unit, a memory 51 as a storage unit, a communication unit 59, a pressure sensor 31, a pump 32, a valve 33, an oscillation circuit 310 for converting the output from the pressure sensor 31 into a frequency, and a pump drive circuit 320 for driving the pump 32. Furthermore, the transmission and reception unit 40 mounts a transmitting and receiving circuit group 45 controlled by the CPU 100 in addition to the transmitting and receiving antenna group 40E described above.

The display 50 includes an organic electro luminescence (EL) display in this example, and displays information related to blood pressure measurement such as blood pressure measurement results and other information in accordance with a control signal from the CPU 100. It should be noted that the display 50 is not limited to the organic EL display, and may include another type of display such as a liquid crystal display (LCD).

The operation unit 52 includes a push switch in this example, and inputs an operation signal corresponding to the user's instructions to start or stop blood pressure measurement into the CPU 100. It should be noted that the operation unit 52 is not limited to the push switch, and may be, for example, a pressure-sensitive (resistive) or proximity (capacitive) touch panel switch. In addition, the operation unit 52 may include a microphone (not shown) to input a blood pressure measurement start instructions in response to the user's voice.

The memory 51 non-transitorily stores data of a program for controlling the sphygmomanometer 1, data used for controlling the sphygmomanometer 1, setting data for setting various functions of the sphygmomanometer 1, data of measurement results of blood pressure values, and the like. In addition, the memory 51 is used as a work memory or the like when a program is executed.

The CPU 100 executes various functions as a control unit in accordance with a program for controlling the sphygmomanometer 1 stored in the memory 51. For example, when blood pressure measurement is performed by the oscillometric method, the CPU 100 performs control to drive the pump 32 (and the valve 33) based on a signal from the pressure sensor 31 in response to instructions to start blood pressure measurement from the operation unit 52. In addition, the CPU 100 performs control to calculate the blood pressure value based on the signal from the pressure sensor 31 in this example.

The communication unit 59 is controlled by the CPU 100 to transmit predetermined information to an external device via the network 900, receive information from an external device via the network 900, and to deliver the information to the CPU 100. The communication via the network 900 may be wireless or wired. In this embodiment, the network 900 is the Internet, but is not limited thereto, and may be another type of network such as a hospital local area network (LAN), or may be one-to-one communication using a USB cable or the like. The communication unit 59 may include a micro USB connector.

The pump 32 and the valve 33 are connected to the pressing cuff 21 via the air pipe 39, and the pressure sensor 31 is connected to the pressing cuff 21 via the air pipe 38. It should be noted that the air pipes 39 and 38 may be one common pipe. The pressure sensor 31 detects the pressure in the pressing cuff 21 via the air pipe 38. The pump 32 includes a piezoelectric pump in this example and supplies air as a fluid for pressurization to the pressing cuff 21 through the air pipe 39 in order to raise the pressure in the pressing cuff 21 (cuff pressure). The valve 33 is mounted on the pump 32, and is configured to be controlled in opening/closing as the pump 32 is turned on/off. That is, when the pump 32 is turned on, the valve 33 closes and air is filled into the pressing cuff 21, while when the pump 32 is turned off, the valve 33 opens and the air in the pressing cuff 21 is discharged into the atmosphere through the air pipe 39. It should be noted that the valve 33 has a function of a check valve so that the discharged air does not flow back. The pump drive circuit 320 drives the pump 32 based on a control signal supplied from the CPU 100.

The pressure sensor 31 is a piezoresistive pressure sensor in this example, and detects the pressure of the belt 20 (pressing cuff 21), a pressure with the atmospheric pressure as a reference (zero) in this example, through the air pipe 38 to output the detected result as a time-series signal. The oscillation circuit 310 oscillates based on an electrical signal value based on a change in electrical resistance due to the piezoresistive effect from the pressure sensor 31, and outputs a frequency signal having a frequency corresponding to the electrical signal value of the pressure sensor 31 to the CPU 100. In this example, the output of pressure sensor 31 is used for controlling the pressure of the pressing cuff 21, and for calculating the blood pressure value (including systolic blood pressure (SBP) and diastolic blood pressure (DBP)) by the oscillometric method.

The battery 53 supplies power to elements mounted on the main body 10, in this example, to each element of the CPU 100, the pressure sensor 31, the pump 32, the valve 33, the display 50, the memory 51, the communication unit 59, the oscillation circuit 310, and the pump drive circuit 320. In addition, the battery 53 also supplies power to the transmitting and receiving circuit group 45 of the transmission and reception unit 40 through the wiring line 71. This wiring line 71 is provided to extend between the main body 10 and the transmission and reception unit 40 along the longitudinal direction X of the belt 20 in a state of being sandwiched between the strip 23 and the pressing cuff 21 of the belt 20 together with the signal wiring line 72.

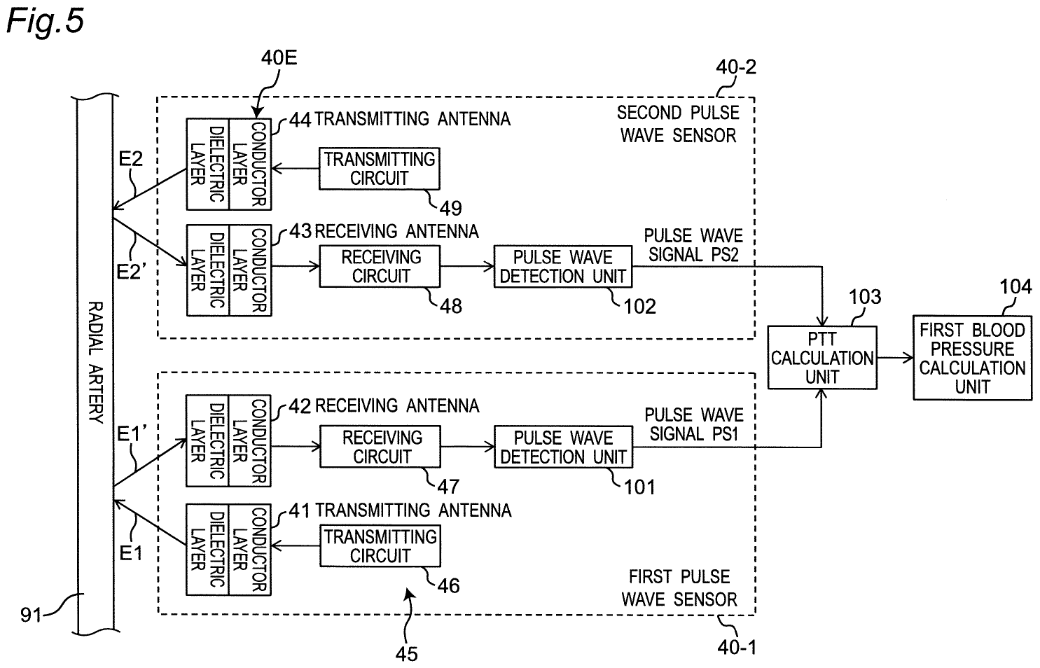

The transmitting and receiving circuit group 45 of the transmission and reception unit 40 includes transmitting circuits 46 and 49 connected to the transmitting antennas 41 and 44, respectively, and receiving circuits 47 and 48 connected to the receiving antennas 42 and 43, respectively. As shown in FIG. 5, the transmitting circuits 46 and 49 emit radio waves E1 and E2 at a frequency of 24 GHz band in this example via the transmitting antennas 41 and 44 connected thereto during operation, respectively. The receiving circuits 47 and 48 receive the radio waves E1′ and E2′ reflected by the left wrist 90 (more precisely, the portion corresponding to the radial artery 91) as the measurement site via the receiving antennas 42 and 43, respectively, to detect and amplify them.

As described in detail below, the pulse wave detection units 101 and 102 shown in FIG. 5 acquire pulse wave signals PS1 and PS2 representing the pulse waves of the radial artery 91 passing through the left wrist 90 based on the outputs of the receiving circuits 47 and 48, respectively. Furthermore, the PTT calculation unit 103 as a time difference acquisition unit acquires a time difference between the pulse wave signals PS1 and PS2 acquired by the two sets of pulse wave detection units 101 and 102, respectively, as a pulse transit time (PTT). In addition, the first blood pressure calculation unit 104 calculates a blood pressure value based on the pulse transit time acquired by the PTT calculation unit 103 by using a predetermined correspondence formula between the pulse transit time and the blood pressure. Here, the pulse wave detection units 101 and 102, the PTT calculation unit 103, and the first blood pressure calculation unit 104 are achieved by the CPU 100 executing a predetermined program. The transmitting antenna 41, the receiving antenna 42, the transmitting circuit 46, the receiving circuit 47, and the pulse wave detection unit 101 constitute a first pulse wave sensor 40-1 as a first set of pulse wave measurement device. The transmitting antenna 44, the receiving antenna 43, the transmitting circuit 49, the receiving circuit 48, and the pulse wave detection unit 102 constitute a second pulse wave sensor 40-2 as a second set of pulse wave measurement device.

In the mounted state, as shown in FIG. 8A, in the longitudinal direction of the left wrist 90 (corresponding to the width direction Y of the belt 20), the first set of transmitting and receiving antenna pair (41, 42) corresponds to the upstream side portion 91u of the radial artery 91 passing through the left wrist 90, while the second set of transmitting and receiving antenna pair (44, 43) corresponds to the downstream side portion 91d of the radial artery 91. The signal acquired by the first set of transmitting and receiving antenna pair (41, 42) represents a change in the distance between the upstream side portion 91u of the radial artery 91 and the first set of transmitting and receiving antenna pair (41, 42) accompanying a pulse wave (which causes expansion and contraction of a blood vessel). The signal acquired by the second set of transmitting and receiving antenna pair (44, 43) represents a change in the distance between the downstream side portion 91d of the radial artery 91 and the second set of transmitting and receiving antenna pair (44, 43) accompanying a pulse wave. The pulse wave detection unit 101 of the first pulse wave sensor 40-1 and the pulse wave detection unit 102 of the second pulse wave sensor 40-2 output in time series the first pulse wave signal PS1 and the second pulse wave signal PS2 each having a mountain-shaped waveform as shown in FIG. 8B based on the outputs of the receiving circuits 47 and 48, respectively.

In this example, the reception levels of the receiving antennas 42 and 43 are about 1 μW (−30 dBm in decibel value with reference to 1 mW). The output levels of the receiving circuits 47 and 48 are about 1 volt. In addition, the respective peaks A1 and A2 of the first pulse wave signal PS1 and the second pulse wave signal PS2 are approximately 100 mV to 1 volt.

It should be noted that assuming that the pulse wave velocity (PWV) of the blood flow of the radial artery 91 is in the range of 1000 cm/s to 2000 cm/s, since the substantial space D between the first pulse wave sensor 40-1 and the second pulse wave sensor 40-2 is 20 mm, the time difference Δt between the first pulse wave signal PS1 and the second pulse wave signal PS2 is in the range of 1.0 ms to 2.0 ms.

In the above example, the case is described where there are two sets of transmitting and receiving antenna pairs, but three or more sets of transmitting and receiving antenna pairs may be used.

(Configuration and Operation of Blood Pressure Measurement by the Oscillometric Method)

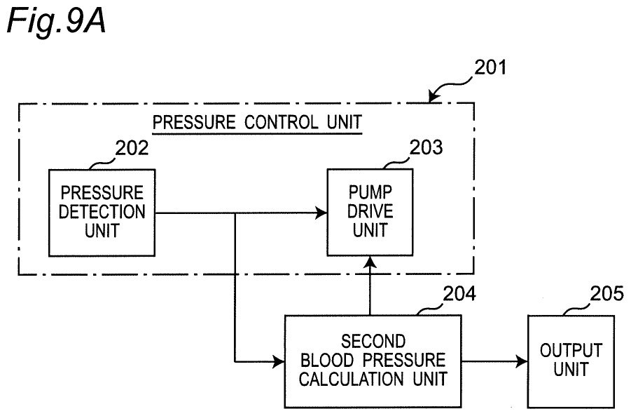

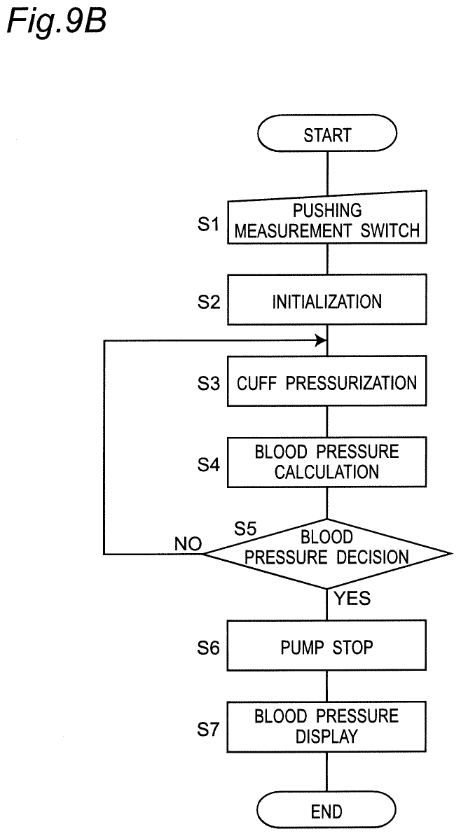

FIG. 9A illustrates a block configuration implemented by a program for performing the oscillometric method in the sphygmomanometer 1.

In this block configuration, roughly, a pressure control unit 201, a second blood pressure calculation unit 204, and an output unit 205 are mounted.

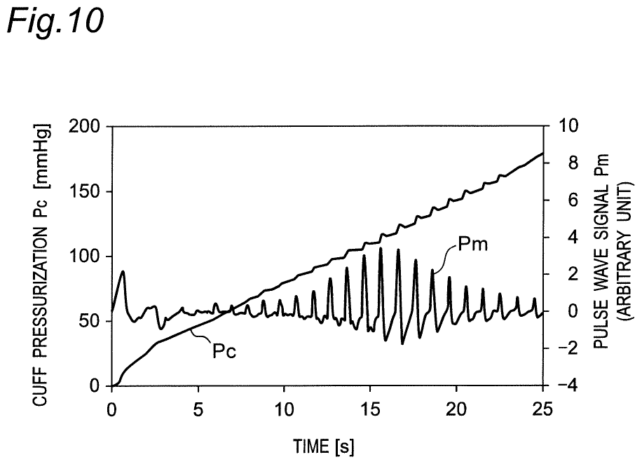

The pressure control unit 201 further includes a pressure detection unit 202 and a pump drive unit 203. The pressure detection unit 202 processes the frequency signal input from the pressure sensor 31 through the oscillation circuit 310, and performs processing for detecting the pressure in the pressing cuff 21 (cuff pressure). The pump drive unit 203 performs processing for driving the pump 32 and the valve 33 through the pump drive circuit 320 based on the detected cuff pressure Pc (see FIG. 10). Thus, the pressure control unit 201 supplies air to the pressing cuff 21 at a predetermined pressurizing speed to control the pressure.

The second blood pressure calculation unit 204 acquires the fluctuation component of the arterial volume included in the cuff pressure Pc as a pulse wave signal Pm (see FIG. 10), and applies a known algorithm by the oscillometric method based on the acquired pulse wave signal Pm to calculate a blood pressure value (systolic blood pressure SBP and diastolic blood pressure DBP). When the calculation of the blood pressure value is completed, the second blood pressure calculation unit 204 stops the processing of the pump drive unit 203.

The output unit 205 performs processing for displaying the calculated blood pressure values (systolic blood pressure SBP and diastolic blood pressure DBP) on the display 50 in this example.

FIG. 9B illustrates an operation flow (flow of blood pressure measurement method) when the sphygmomanometer 1 performs blood pressure measurement by the oscillometric method. The belt 20 of the sphygmomanometer 1 is assumed to be mounted in advance so as to wind around the left wrist 90.

When the user instructs blood pressure measurement by oscillometric method with the push switch as the operation unit 52 provided in the main body 10 (step S1), the CPU 100 starts operation to initialize the processing memory area (step S2). In addition, the CPU 100 turns off the pump 32 via the pump drive circuit 320, opens the valve 33, and discharges the air in the pressing cuff 21. Subsequently, control is performed to set the current output value of the pressure sensor 31 as a value corresponding to the atmospheric pressure (0 mmHg adjustment).

Subsequently, the CPU 100 operates as the pump drive unit 203 of the pressure control unit 201 to close the valve 33, and then drives the pump 32 via the pump drive circuit 320 to perform control to send air to the pressing cuff 21. Thus, the pressing cuff 21 is inflated and the cuff pressure Pc (see FIG. 10) is gradually increased to compress the left wrist 90 as the measurement site (step S3 in FIG. 9B).

In this pressurization process, in order to calculate the blood pressure value, the CPU 100 works as the pressure detection unit 202 of the pressure control unit 201, monitors the cuff pressure Pc with the pressure sensor 31, and acquires, as a pulse wave signal Pm as illustrated in FIG. 10, the fluctuation component of the arterial volume generated in the radial artery 91 of the left wrist 90.

Next, in step S4 in FIG. 9B, the CPU 100 acts as a second blood pressure calculation unit, and applies a known algorithm by oscillometric method based on the pulse wave signal Pm acquired at this time to attempt the calculation of blood pressure values (systolic blood pressure SBP and diastolic blood pressure DBP).

At this time, if the blood pressure value cannot be calculated yet because of insufficient data (NO in step S5), unless the cuff pressure Pc reaches the upper limit pressure (for safety, for example, 300 mmHg is predetermined), the processing of steps S3 to S5 is repeated.

If the blood pressure value can be calculated in this manner (YES in step S5), the CPU 100 stops the pump 32, opens the valve 33, and performs control to discharge the air in the pressing cuff 21 (step S6). Then, lastly, the CPU 100 works as the output unit 205, displays the measurement result of the blood pressure value on the display 50, and records the measurement result in the memory 51 (step S7).

It should be noted that the calculation of the blood pressure value may be performed not only in the pressurization process, but also in the depressurization process.

(Operation of Blood Pressure Measurement Based on Pulse Transit Time)

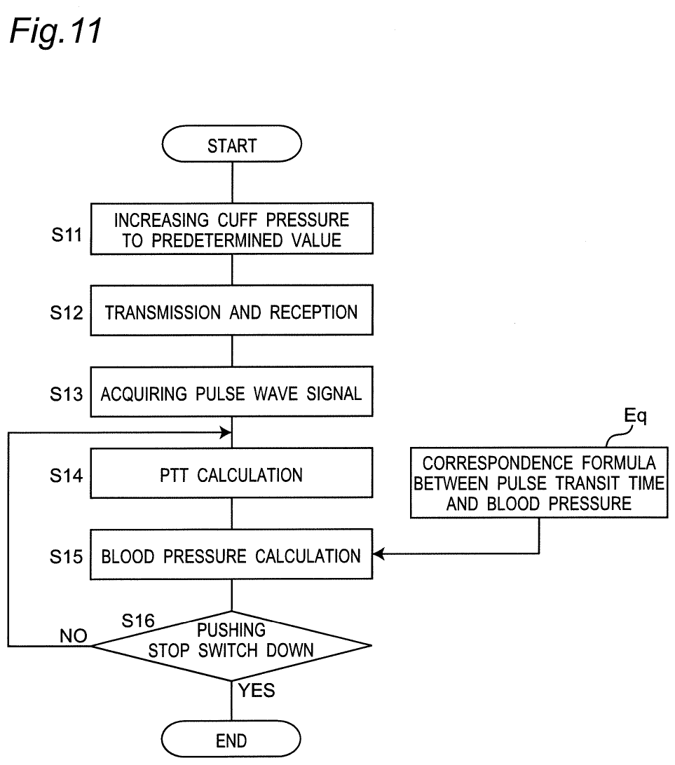

FIG. 11 illustrates an operation flow according to the biological information measurement method, pulse wave measurement method, and blood pressure measurement method of one embodiment of the present invention; the operation flow including: the sphygmomanometer 1 performing pulse wave measurement, acquiring a pulse transit time (PTT), and performing blood pressure measurement (estimation) based on the pulse transit time. The belt 20 of the sphygmomanometer 1 is assumed to be mounted in advance so as to wind around the left wrist 90.

When the user gives an instruction to perform the PTT-based blood pressure measurement with a push switch as the operation unit 52 provided on the main body 10, the CPU 100 starts operation. That is, the CPU 100 closes the valve 33 and drives the pump 32 via the pump drive circuit 320, and performs control to send air to the pressing cuff 21 to expand the pressing cuff 21 and to increase the cuff pressure Pc (see FIG. 8A) to a predetermined value (step S11 in FIG. 11). In this example, in order to lighten the physical burden on the user, the pressure is kept to the degree enough to have the belt 20 in close contact with the left wrist 90 (for example, about 5 mmHg). Thus, the transmitting and receiving antenna group 40E is securely caused to abut on the palmar surface 90a of the left wrist 90, so that no gap is generated between the palmar surface 90a and the transmitting and receiving antenna group 40E. It should be noted that the step S11 may be omitted.

At this time, as shown in FIG. 8A, in each of the first pulse wave sensor 40-1 and the second pulse wave sensor 40-2, (the second surface 402b of) the dielectric layer 402 of the transmitting and receiving antenna group 40E abuts on the palmar surface 90a of the left wrist 90. Therefore, in each of the first pulse wave sensor 40-1 and the second pulse wave sensor 40-2, the conductor layer 401 faces the palmar surface 90a of the left wrist 90, and the dielectric layer 402 keeps the distance between the palmar surface 90a of the left wrist 90 and the conductor layer 401 (distance in the thickness direction) constant. In addition, as described above, in the longitudinal direction of the left wrist 90 (corresponding to the width direction Y of the belt 20), the first set of transmitting and receiving antenna pair (41, 42) corresponds to the upstream side portion 91u of the radial artery 91 passing through the left wrist 90, while the second set of transmitting and receiving antenna pair (44, 43) corresponds to the downstream side portion 91d of the radial artery 91.

Next, in the mounted state, as shown in step S12 in FIG. 11, the CPU 100 controls transmission and reception in each of the first pulse wave sensor 40-1 and the second pulse wave sensor 40-2 shown in FIG. 5. Specifically, as shown in FIG. 8A, in the first pulse wave sensor 40-1, the transmitting circuit 46 emits a radio wave E1 toward the upstream side portion 91u of the radial artery 91 via the transmitting antenna 41, that is, from the conductor layer 401 through the dielectric layer 402 (or the gap existing on the side of the dielectric layer 402). Along with this, the receiving circuit 47 receives the radio wave E1′ reflected by the upstream side portion 91u of the radial artery 91 with the conductor layer 401 via the receiving antenna 42, that is, through the dielectric layer 402 (or the gap existing on the side of the dielectric layer 402), and detects and amplifies the radio wave E1′. In addition, in the second pulse wave sensor 40-2, the transmitting circuit 49 emits a radio wave E2 toward the downstream side portion 91d of the radial artery 91 via the transmitting antenna 44, that is, from the conductor layer 401 through the dielectric layer 402 (or the gap existing on the side of the dielectric layer 402). Along with this, the receiving circuit 48 receives the radio wave E2′ reflected by the downstream side portion 91d of the radial artery 91 with the conductor layer 401 via the receiving antenna 43, that is, through the dielectric layer 402 (or the gap existing on the side of the dielectric layer 402), and detects and amplifies the radio wave E2′.

Next, as shown in step S13 in FIG. 11, the CPU 100 works as the pulse wave detection units 101 and 102 in the first pulse wave sensor 40-1 and the second pulse wave sensor 40-2 shown in FIG. 5 and acquires pulse wave signals PS1 and PS2 as shown in FIG. 8B, respectively. That is, in the first pulse wave sensor 40-1, the CPU 100 works as the pulse wave detection unit 101 and acquires a pulse wave signal PS1 representing the pulse wave of the upstream side portion 91u of the radial artery 91 from the output in the vasodilation phase and the output in the vasoconstriction phase of the receiving circuit 47. In addition, in the second pulse wave sensor 40-2, the CPU 100 works as the pulse wave detection unit 102 and acquires a pulse wave signal PS2 representing the pulse wave of the downstream side portion 91d of the radial artery 91 from the output in the vasodilation phase and the output in the vasoconstriction phase of the receiving circuit 48.

Next, as shown in step S14 in FIG. 11, the CPU 100 works as a PTT calculation unit 103 as a time difference acquisition unit, and acquires a time difference between the pulse wave signal PS1 and the pulse wave signal PS2 as a pulse transit time (PTT). More specifically, in this example, a time difference Δt between the peak A1 of the first pulse wave signal PS1 and the peak A2 of the second pulse wave signal PS2 shown in FIG. 8B is acquired as the pulse transit time (PTT).

Thereafter, as shown in step S15 in FIG. 11, the CPU 100 works as a first blood pressure calculation unit, and calculates (estimates) the blood pressure based on the pulse transit time (PTT) acquired in step S14 by using the predetermined correspondence formula Eq between the pulse transit time and the blood pressure. Here, when pulse transit time is represented as DT and blood pressure is represented as EBP, the predetermined correspondence formula Eq between pulse transit time and blood pressure is provided as a known fractional function including the term of 1/DT², such as shown in a formula:

EBP=α/DT²+β (Eq. 1)

(where each of α and β represents a known coefficient or constant) (see, for example, JP H10-201724 A).

It should be noted that as a predetermined correspondence formula Eq between pulse transit time and blood pressure, another known correspondence formula such as a formula including the term of 1/DT and the term of DT may be used in addition to the term of 1/DT², such as shown in another formula:

EBP=α/DT²+β/DT+γDT+δ (Eq. 2)

(where each of α, β, γ, and δ represents a known coefficient or constant).

When the blood pressure is calculated (estimated) in this way, as described above, in each of the first pulse wave sensor 40-1 and the second pulse wave sensor 40-2, the dielectric layer 402 keeps the distance between the palmar surface 90a of the left wrist 90 and the conductor layer 401 constant. In addition, due to the dielectric layer 402 interposed between the palmar surface 90a of the left wrist 90 and the conductor layer 401, it is less likely to be affected by fluctuations in the dielectric constant of the living body (the relative permittivity of the living body varies in the range of about 5 to 40). In addition, since room between the palmar surface 90a of the left wrist 90 and the conductor layer 401 can be made, the range (area) irradiated with radio waves on the palmar surface 90a of the left wrist 90 can be expanded as compared with the case where the conductor layer 401 is in direct contact with the palmar surface 90a of the left wrist 90. Therefore, even if the mounting position of the conductor layer 401 is slightly shifted from directly above the radial artery 91, the signal reflected by the radial artery 91 can be stably received. As a result, the signal levels received by the respective receiving circuits 47 and 48 are stabilized, and the pulse wave signals PS1 and PS2 as biological information can be acquired with high precision. As a result, the pulse transit time (PTT) can be acquired with high precision, and therefore, the blood pressure value can be calculated (estimated) with high precision. It should be noted that the measurement result of the blood pressure value is displayed on the display 50 and recorded in the memory 51.

In this example, if measurement stop is not instructed by the push switch as the operation unit 52 in step S16 in FIG. 11 (NO in step S16), the calculation of the pulse transit time (PTT) (step S14 in FIG. 11) and the calculation (estimation) of the blood pressure (step S15 in FIG. 11) are periodically repeated every time the first and second pulse wave signals PS1 and PS2 are input according to the pulse wave. The CPU 100 updates and displays the measurement result of the blood pressure value on the display 50, and accumulates and records the measurement result in the memory 51. Then, if measurement stop is instructed in step S16 in FIG. 11 (YES in step S16), the measurement operation is ended.

According to the sphygmomanometer 1, the blood pressure measurement based on the pulse transit time (PTT) allows blood pressure to be measured continuously over a long period of time with a reduced physical burden on the user.

In addition, according to the sphygmomanometer 1, the blood pressure measurement (estimation) based on pulse transit time and the blood pressure measurement by the oscillometric method can be performed using a common belt 20 with an integrated device. Therefore, the convenience of the user can be enhanced. For example, in general, when blood pressure measurement (estimation) based on pulse transit time (PTT) is performed, it is necessary to appropriately calibrate the correspondence formula Eq between the pulse transit time and the blood pressure (in the above example, update the values of the coefficients α, β, and the like based on the actually measured pulse transit time and the blood pressure value). Here, according to the sphygmomanometer 1, the blood pressure measurement by the oscillometric method can be performed with the same apparatus, and the correspondence formula Eq can be calibrated based on the result, so that the convenience of the user can be enhanced. In addition, a rapid rise in blood pressure can be captured by the PTT method (blood pressure measurement based on pulse transit time) that can be continuously measured even though the precision is low, and with the rapid rise in blood pressure as a trigger, measurement by a more precise oscillometric method can be started.

(First Modification)

In the above examples, as illustrated in FIG. 6, the relative permittivity of the dielectric layer 402 constituting the transmitting and receiving antenna group 40E is assumed to be uniformly set to εᵣ≈3.0, but the present invention is not limited to this. The relative permittivity (εᵣ) of the dielectric layer 402 has only to be set in the range of 1 to 5. In that case, the relative permittivity (εᵣ) of the dielectric layer 402 and the relative permittivity of the left wrist 90 (within a range of about 5 to 40) increase in this order. Therefore, power reflection at the interface between the left wrist 90 and the dielectric layer 402 is reduced. As a result, the SN ratio (signal-to-noise ratio) of the received signal is increased, and the pulse wave signals PS1 and PS2 as biological information can be precisely measured.

Furthermore, as illustrated in FIG. 7, it is desirable that the relative permittivity (εᵣ) of the dielectric layer 402 gradually increases from the first surface 402a on the side along the conductor layer 401 toward the second surface 402b (the surface on the side abutting on the palmar surface 90a of the left wrist 90 in the mounted state) on the side opposite to that of the first surface 402a. In the example in FIG. 7, the dielectric layer 402 includes a three-layer structure, provided in order from the first surface 402a toward the second surface 402b, of a silicone layer (relative permittivity εᵣ≈2.4) 402-1, a polycarbonate layer (relative permittivity εᵣ≈3.0) 402-2, and a nylon layer (relative permittivity εᵣ≈4.2) 402-3. That is, the relative permittivity (Er) of the dielectric layer 402 increases stepwise from the first surface 402a toward the second surface 402b. Thus, power reflection at the interface between the left wrist 90 and the dielectric layer 402 is reduced. As a result, the SN ratio (signal-to-noise ratio) of the received signal is increased, and the pulse wave signals PS1 and PS2 as biological information can be precisely measured. It should be noted that the dielectric layer 402 is not limited to a three-layer structure, and may be configured in more layers. In addition, the relative permittivity of the dielectric layer 402 may increase continuously from the first surface 402a toward the second surface 402b instead of stepwise.

It should be noted that there are individual differences in the shape of the measurement site (wrist). A person with an almost flat measurement site can be measured with sufficiently high precision even without flexibility. Flexibility allows measurement with high precision regardless of the shape of the measurement site.

(Second Modification)

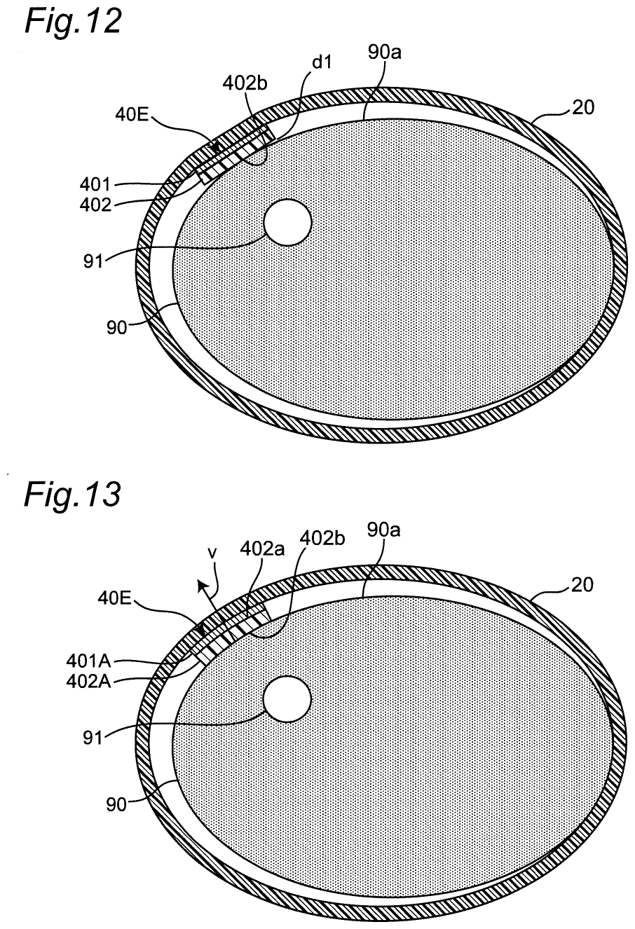

In the above examples, the dielectric layer 402 constituting the transmitting and receiving antenna group 40E is assumed to be made of polycarbonate, that is, a material having relatively poor flexibility. Therefore, as illustrated in FIG. 12, a gap d1 may occur between the palmar surface 90a of the left wrist 90 and the end portion of the second surface 402b of the dielectric layer 402. Thus, in this example, the conductor layer 401 and the dielectric layer 402 are assumed to have flexible structure that can be deformed along the palmar surface 90a of the left wrist 90 as a whole. For example, the dielectric layer 402A shown in FIG. 13 is assumed to be made of a material having relatively high flexibility such as silicone resin (relative permittivity εᵣ≈2.4) or nylon (relative permittivity εᵣ≈4.2). The conductor layer 401A is assumed to be made of, for example, a metal layer having a thickness of about several μm to 30 μm deposited on the first surface 402a of the dielectric layer 402A. Thus, the conductor layer 401A and the dielectric layer 402A can be deformed along the palmar surface 90a of the left wrist 90 as a whole due to flexibility. Therefore, even if the palmar surface 90a of the left wrist 90 is curved, a gap is unlikely to occur between the palmar surface 90a of the left wrist 90 and the second surface 402b of the dielectric layer 402A. As a result, the distance between the palmar surface 90a of the left wrist 90 and the conductor layer 401A (distance in the thickness direction v) is kept constant. In addition, since no gap occurs between the palmar surface 90a of the left wrist 90 and the second surface 402b of the dielectric layer 402A, no radio wave propagation loss due to such a gap occurs. Therefore, the received signal level is further stabilized, and the pulse wave signals PS1 and PS2 as biological information can be measured with high precision.

(Third Modification)

In addition, the dielectric layer 402 constituting the transmitting and receiving antenna group 40E may be at least partially made of a hygroscopic cloth. For example, in the dielectric layer 402 having a three-layer structure shown in FIG. 7, the nylon layer 402-3 may be made of hygroscopic cloth. Thus, even if the subject sweats on the left wrist 90, the sweat is absorbed by the portion made of hygroscopic cloth of the dielectric layer 402 (nylon layer 402-3), and is prevented from staying between the left wrist 90 and the dielectric layer 402. As a result, discomfort of the user mounted with the sphygmomanometer 1 (including the transmitting and receiving antenna group 40E) is reduced.

(Fourth Modification)

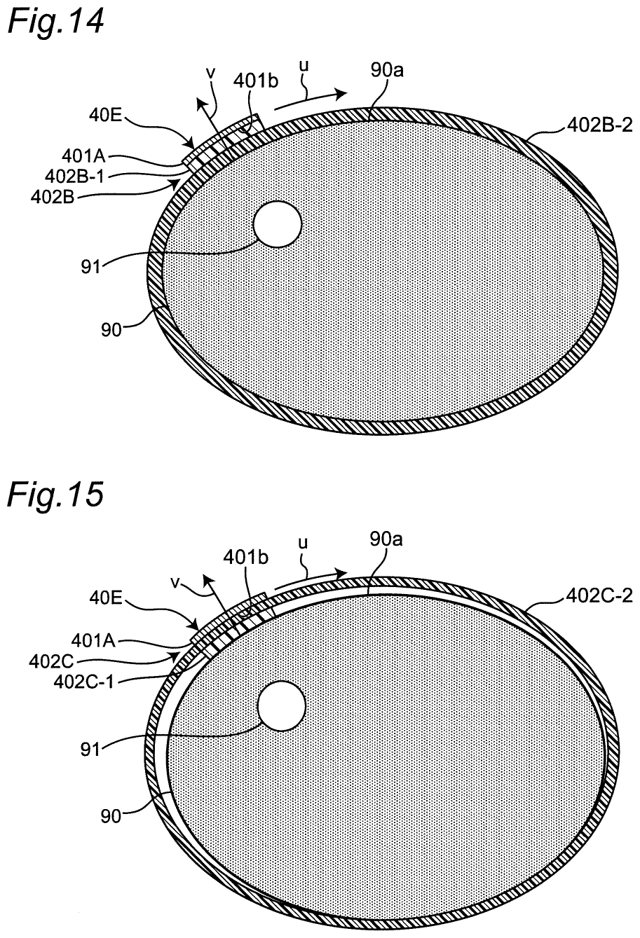

In the above examples, the case is described where all of the dielectric layers 402 constituting the transmitting and receiving antenna group 40E have a square pattern shape. However, the present invention is not limited thereto. For example, as shown in FIG. 14, the dielectric layer 402B may be configured by stacking, in the thickness direction v, a specific portion 402B-1 having a square pattern shape provided in a range corresponding to the facing surface 401b of the conductor layer 401A and a strip-shaped layer portion 402B-2 extending in a strip shape beyond the range occupied by the specific portion 402B-1. In this example, the strip-shaped layer portion 402B-2 is configured in an annular shape so as to wind around the left wrist 90. In this case, the specific portion 402B-1 is assumed to be made of, for example, a silicone resin having a thickness of about 2 mm (relative permittivity εᵣ≈2.4). The strip-shaped layer portion 402B-2 is assumed to be made of, for example, nylon having a thickness of about 1 mm to 2 mm (relative permittivity εᵣ≈4.2).

According to this configuration, the user's winding around the left wrist 90 with the strip-shaped layer portion 402B-2 of the dielectric layer 402B mounts the transmitting and receiving antenna group 40E on the left wrist 90. That is, the strip-shaped layer portion 402B-2 can constitute a part of the belt 20 that winds around the left wrist 90 (for example, an inner cloth that covers the inner peripheral surface 20a of the belt 20). In addition, for example, in a case of a simple configuration where the pressing cuff 21 is omitted in the belt 20 and only blood pressure measurement based on the pulse transit time (PTT) is performed, the belt 20 can be entirely constituted by the strip-shaped layer portion 402B-2.

In this example, it is particularly desirable that the strip-shaped layer portion 402B-2 is made of a hygroscopic cloth. In that case, even if sweat of the living body occurs on the left wrist 90, the sweat is absorbed by the strip-shaped layer portion 402B-2 (made of hygroscopic cloth) of the dielectric layer 402B and is prevented from staying between the outer peripheral surface of the left wrist 90 and the inner peripheral surface of the strip-shaped layer portion 402B-2. As a result, discomfort of the user is reduced.

It should be noted that as in the dielectric layer 402C shown in FIG. 15, in the thickness direction v, the stacking order of the specific portion 402C-1 and the strip-shaped layer portion 402C-2 may be reversed from the stacking order of the specific portion 402B-1 and the strip-shaped layer portion 402B-2 in FIG. 14. In this case, the strip-shaped layer portion 402C-2 is assumed to be made of a silicone resin (relative permittivity εᵣ≈2.4) having a thickness of, for example, about 1 mm to 2 mm. The specific portion 402C-1 is assumed to be made of nylon (relative permittivity εr≈4.2) having a thickness of, for example, about 2 mm. Also in this case, substantially the same action and effect as those in FIG. 14 can be obtained.

(Fifth Modification)

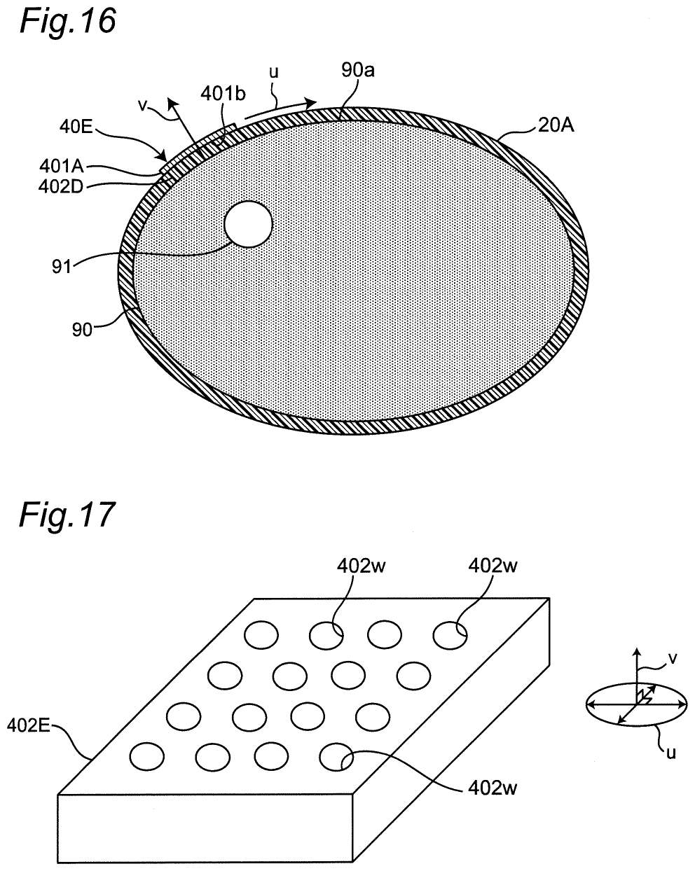

In the above examples, the case is described where the dielectric layer 402 constituting the transmitting and receiving antenna group 40E has a square pattern shape corresponding at least partially to the facing surface 401b of the conductor layers 401 and 401A. However, the present invention is not limited thereto. For example, in a case of a simple configuration where the pressing cuff 21 is omitted and only blood pressure measurement based on the pulse transit time (PTT) is performed, as shown in FIG. 16, the dielectric layer 402D constituting the transmitting and receiving antenna group 40E may be made only of a portion corresponding to the facing surface 401b of the conductor layer 401A of the belt 20A extending in a strip shape so as to wind around the left wrist 90. The belt 20A is assumed to be made of nylon (relative permittivity εᵣ≈4.2) having a thickness of, for example, about 1 mm to 2 mm. Also in this case, the user's winding around the left wrist 90 with the belt 20A allows the transmitting and receiving antenna group 40E to be mounted on the left wrist 90. Furthermore, in the configuration in FIG. 16, the configuration of the dielectric layer 402D can be simplified as compared with the case where the specific portions 402B-1 and 401C-1 are provided as shown in FIGS. 14 and 15.

(Sixth Modification)

In the above examples, the case is described where the relative permittivity of the portion corresponding to the facing surface 401b of the conductor layer of each of the dielectric layers 402, 402A, 402B, 402C, and 402D is uniform in the surface direction u. However, the present invention is not limited thereto. For example, a dielectric layer 402E as shown in FIG. 17 may be used instead of the dielectric layer 402 in FIG. 6. As an example of the cavity, the dielectric layer 402E includes, dispersedly in the surface direction u, a plurality of through holes 402w, 402w, . . . each having a circular cross section, penetrating the dielectric layer 402E in the thickness direction v. The material of the dielectric layer 402E is the same polycarbonate (relative permittivity εᵣ≈3.0) as the material of the dielectric layer 402 in FIG. 6. The relative permittivity of the through holes 402w, 402w, . . . is substantially equal to 1, and is smaller than the relative permittivity of the material of the dielectric layer 402 itself (εᵣ≈3.0). Thus, the effective relative permittivity (εr) as a whole of the dielectric layer 402E is set lower than the relative permittivity (εᵣ≈3.0) of the polycarbonate itself.

In this example, the area of the dielectric layer 402E in the surface direction u is set to 10 mm², and the dimension in the thickness direction v is set to 2 mm. The area in the surface direction u of each through hole 402w is set to 2 mm² (therefore, the diameter is about 0.5 mm). When the number of through holes 402w is, for example, 10, the effective relative permittivity as a whole of the dielectric layer 402E is εᵣ≈2.6.

Varying the number of these through holes 402w, 402w, . . . or the density in the surface direction u allows the effective relative permittivity as a whole of the dielectric layer 402E to be variably set. Therefore, the degree of freedom for setting the effective relative permittivity as a whole of the dielectric layer 402E is increased.

It should be noted that in order to variably set the effective relative permittivity as a whole of the dielectric layer 402, a plurality of minute spherical cavities may be provided inside the dielectric layer 402 in a dispersed manner in the surface direction u and the thickness direction v, for example.

(Effect of Interposing Dielectric Layer)

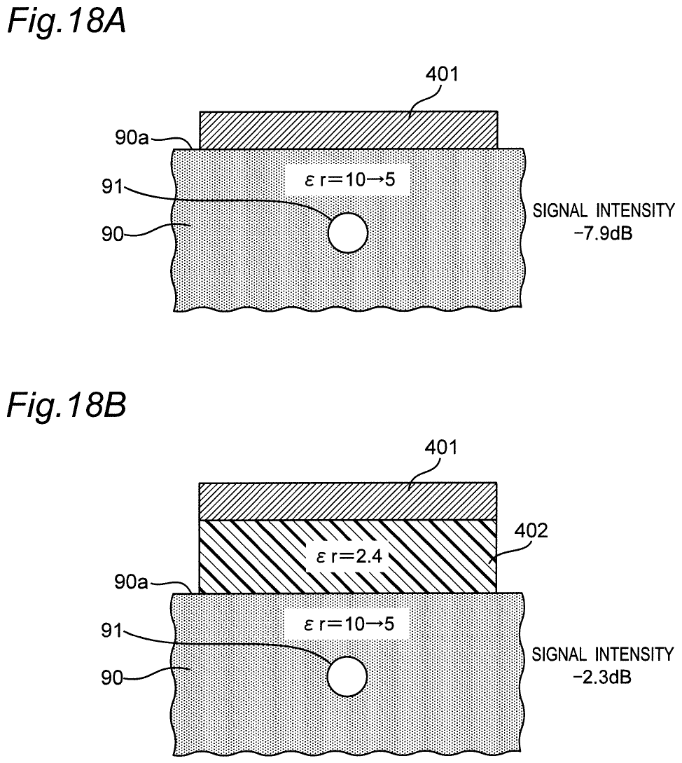

FIG. 18A illustrates a mode in which no dielectric layer is interposed between the palmar surface 90a of the left wrist 90 and the conductor layer 401 and the conductor layer 401 is in direct contact with the palmar surface 90a of the left wrist 90. According to the present inventor's electromagnetic field analysis, it was understood that when, in this mode, the relative permittivity (Er) in the vicinity of the palmar surface 90a of the left wrist 90 varies, for example, from 10 to 5, the intensity of the received signal decreases by 7.9 dB. On the other hand, FIG. 18B illustrates a mode in which a dielectric layer (silicone resin, 2 mm in thickness, relative permittivity εᵣ≈2.4) 402 is interposed between the palmar surface 90a of the left wrist 90 and the conductor layer 401 according to the present invention. According to the present inventor's electromagnetic field analysis, it was understood that when, in this mode, the relative permittivity (Er) in the vicinity of the palmar surface 90a of the left wrist 90 varies, for example, from 10 to 5, the intensity of the received signal only decreases by 2.3 dB. As a result, it was confirmed that interposing a dielectric layer between the measurement site of the living body and the conductor layer constituting the antenna as in the present invention makes it less susceptible to fluctuations in the dielectric constant of the living body (the relative permittivity of the living body varies in the range of about 5 to 40) and that the received signal level is stabilized.

(Seventh Modification)

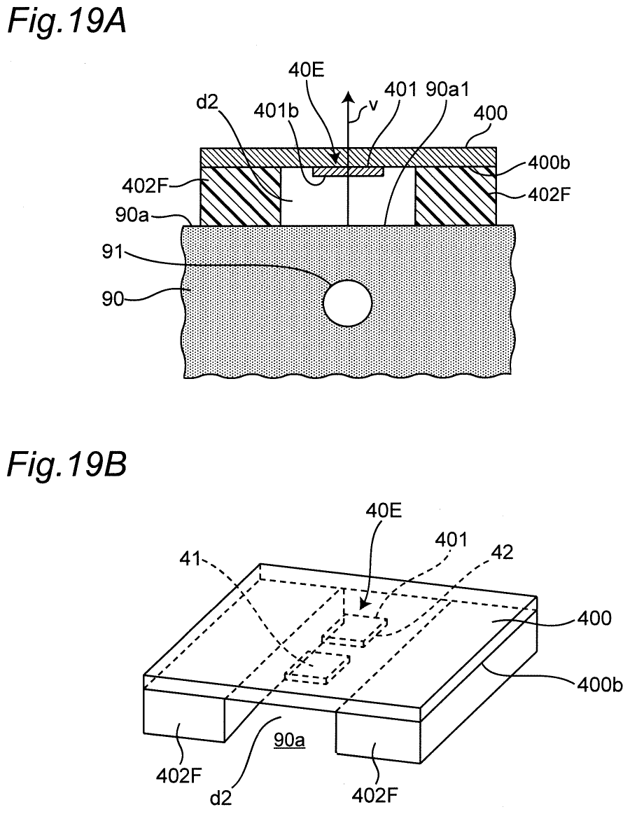

In each of the above examples, a case is described in which the dielectric layer is directly mounted along the facing surface (the surface facing the left wrist 90) 401b of the conductor layer 401 or 401A constituting the transmitting and receiving antenna group 40E. However, the present invention is not limited thereto. For example, as shown in FIGS. 19A and 19B, dielectric layers 402F, 402F may be mounted along the facing surface 400b facing the left wrist 90 of the base material 400 mounting the conductor layer 401 and extending in parallel with the conductor layer 401. As shown in FIG. 19B, in this example, the conductor layer 401 is divided into a transmitting antenna 41 and a receiving antenna 42 that receives a radio wave from the transmitting antenna 41.

In this configuration, in the mounted state where the transmitting and receiving antenna group 40E is mounted on the left wrist 90, the conductor layer 401 is disposed facing the palmar surface 90a of the left wrist 90, and the dielectric layer 402F is disposed between the palmar surface 90a of the left wrist 90 and the facing surface 400b of the base material 400. A gap d2 is formed between a portion 90a1 corresponding to the radial artery 91 of the palmar surface 90a of the left wrist 90 and the facing surface 401b of the conductor layer 401. In this mounted state, the dielectric layer 402F keeps the distance (distance in the thickness direction v) between the palmar surface 90a of the left wrist 90 and (the facing surface 401b of) the conductor layer 401 constant.

In this mounted state, a radio wave is emitted from the transmitting antenna 41 toward the left wrist 90 through the gap d2 (or the dielectric layers 402F present on the sides of the gap d2). The radio wave reflected by the left wrist 90 is received by the receiving antenna 42 through the gap d2 (or the dielectric layers 402F present on the sides of the gap d2). Here, according to this configuration, since the dielectric layer 402F keeps the distance between the palmar surface 90a of the left wrist 90 and the conductor layer 401 (transmitting and receiving antenna pair (41, 42)) constant, the received signal level is stabilized, and biological information can be measured with high precision. In addition, as compared with the above examples, dielectric loss due to the dielectric layer immediately below the conductor layer 401 can be reduced, and the SN ratio of the received signal can be increased. Therefore, the pulse wave signals PS1 and PS2 as biological information can be measured with high precision.

In the above-described embodiment, as shown in FIG. 3, it is assumed that the transmitting antennas 41 and 44 are arranged on both sides in the range occupied by the transmitting and receiving antenna group 40E in the width direction Y, and that the receiving antennas 42 and 43 are arranged between the transmitting antennas 41 and 44. However, the present invention is not limited thereto. The receiving antennas 42 and 43 may be arranged on both sides in the range occupied by the transmitting and receiving antenna group 40E, and the transmitting antennas 41 and 44 may be arranged between the receiving antennas 42 and 43. In this arrangement, the receiving antenna 42 is closer to the transmitting antenna 41 than the receiving antenna 43 in the width direction Y. In addition, the receiving antenna 43 is closer to the transmitting antenna 44 than the receiving antenna 42 in the width direction Y. Therefore, interference between the first set of transmitting and receiving antenna pair (41, 42) and the second set of transmitting and receiving antenna pair (44, 43) can be reduced.

In the above-described embodiment, it is assumed that the conductor layers 401 and 401A are configured such that the transmitting antenna and the receiving antenna that receives a radio wave from the transmitting antenna are separated from each other and divided. However, the present invention is not limited thereto. The conductor layer forming the biometric antenna device may be used as a spatially single transmitting and receiving shared antenna via a known circulator for the emission and reception of radio waves.

In addition, in the above embodiment, the sphygmomanometer 1 is intended to be mounted on the left wrist 90 as a measurement site. However, the present invention is not limited thereto. The measurement site has only to be a site which an artery passes through, may be the right wrist, an upper limb such as an upper arm other than the wrist, and may be a lower limb such as an ankle or thigh.

In addition, in the above embodiments, the CPU 100 mounted on the sphygmomanometer 1 is assumed to work as a pulse wave detection unit, and first and second blood pressure calculation units to perform blood pressure measurement by oscillometric method (operation flow in FIG. 9B) and blood pressure measurement (estimation) based on PTT (operation flow in FIG. 11). However, the present invention is not limited thereto. For example, a substantial computer device such as a smartphone provided outside the sphygmomanometer 1 may work as a pulse wave detection unit and first and second blood pressure calculation units to cause, via the network 900, the sphygmomanometer 1 to perform blood pressure measurement by oscillometric method (operation flow in FIG. 9B) and blood pressure measurement (estimation) based on PTT (operation flow in FIG. 11). In that case, the user can perform an operation such as an instruction to start or stop blood pressure measurement using the operation unit (touch panel, keyboard, mouse, or the like) of the computer device, and can cause the display (organic EL display, LCD, or the like) of the computer device to display information related to blood pressure measurement such as blood pressure measurement results and other information. In that case, in the sphygmomanometer 1, the display 50 and the operation unit 52 may be omitted.

In the above-described embodiments, the sphygmomanometer 1 measures the pulse wave signal, the pulse transit time, and the blood pressure as biological information, but the present invention is not limited thereto. Various other pieces of biological information such as the pulse rate may be measured.

In addition, the present invention may constitute an apparatus including a biometric antenna device, a pulse wave measurement device, or a blood pressure measurement device and further including a functional unit for performing another function. According to this apparatus, biological information can be measured with high precision, and in particular, pulse wave signals can be acquired with high precision as biological information, or the blood pressure value can be calculated (estimated) with high precision. In addition, this apparatus can perform various functions.

As described above, a biometric antenna device of the present disclosure for emitting a radio wave toward a measurement site of a living body or for receiving a radio wave from the measurement site, the biometric antenna device comprises:

a conductor layer configured to face the measurement site for emitting and/or receiving the radio wave; and

a dielectric layer mounted along a facing surface facing the measurement site of the conductor layer or of a base material mounting the conductor layer and extending in parallel with the conductor layer, the dielectric layer having a predetermined relative permittivity,

wherein the dielectric layer keeps a distance between an outer surface of the measurement site and the conductor layer constant, in a mounted state in which a second surface on a side opposite to a side of a first surface on a side along the conductor layer of the dielectric layer abuts on an outer surface of the measurement site.

In the present specification, the “measurement site” may be a trunk in addition to a rod-shaped site such as an upper limb (wrist, upper arm, or the like) or a lower limb (ankle, or the like).

In addition, the “outer surface” of the measurement site refers to a surface exposed to the outside. For example, if the measurement site is a wrist, it refers to the outer peripheral surface of the wrist or a part thereof (for example, the palmar surface corresponding to the palmar side portion in the circumferential direction of the outer peripheral surface).

In addition, the “conductor layer” can be used, for emitting and/or receiving a radio wave, as a transmitting antenna or a receiving antenna, or as a transmitting and receiving shared antenna via a known circulator. The “conductor layer” may be divided into a transmitting antenna and a receiving antenna that receives a radio wave from the transmitting antenna.

In addition, unless otherwise noted, the “predetermined relative permittivity” of the dielectric layer may be uniform over the range in which the dielectric layer occupies space, or may vary depending on the position within a range in which the dielectric layer occupies space.

In addition, a phrase that the dielectric layer “keeps a distance constant” between an outer surface of the measurement site and the conductor layer means that the dielectric layer is a spacer. It should be noted that in the case where the dielectric layer has flexibility, it means acceptable that the “distance” more or less fluctuates due to the bending when it is bent by an external force.

In the biometric antenna device of the present invention, in the mounted state mounted on the measurement site, a second surface of the dielectric layer on a side opposite to a side of the first surface on a side along the conductor layer abuts on the outer surface of the measurement site. In this mounted state, the conductor layer faces the outer surface of the measurement site, and the dielectric layer keeps a distance between the outer surface of the measurement site and the conductor layer (distance in the thickness direction) constant. When the conductor layer is used as a transmitting antenna in this mounted state, a radio wave is emitted from the conductor layer toward the measurement site through the dielectric layer (or a gap present on the side of the dielectric layer). Here, since the dielectric layer keeps the distance between the outer surface of the measurement site and the conductor layer constant, the signal level applied to the measurement site is stabilized. On the other hand, when the conductor layer is used as a receiving antenna, the radio wave reflected by the measurement site is received by the conductor layer through the dielectric layer (or a gap present on the side of the dielectric layer). Here, the dielectric layer keeps the distance between the outer surface of the measurement site and the conductor layer constant. In addition, due to the dielectric layer interposed between the outer surface of the measurement site and the conductor layer (or the base material), it is less likely to be affected by fluctuations in the dielectric constant of the living body (the relative permittivity of the living body varies in the range of about 5 to 40). In addition, since room between the outer surface of the measurement site and the conductor layer can be made, the range (area) of the measurement site irradiated with a radio wave can be expanded as compared with the case where the conductor layer is in direct contact with the outer surface of the measurement site. As a result, the received signal level is stabilized. Therefore, according to this biometric antenna device, biological information can be measured with high precision.

In the biometric antenna device of one embodiment, the conductor layer or the base material and the dielectric layer have flexibility configured to be deformed along an outer surface of the measurement site as a whole.

When the biometric antenna device of this embodiment is mounted on the measurement site of the living body, the conductor layer or the base material and the dielectric layer can be deformed along the outer surface of the measurement site as a whole due to the flexibility. Therefore, even when the outer surface of the measurement site is curved, a gap is unlikely to occur between the outer surface of the measurement site and the second surface of the dielectric layer. As a result, even when the outer surface of the measurement site is curved, the distance between the outer surface of the measurement site and the conductor layer is kept constant. In addition, power reflection at the interface between the measurement site and the dielectric layer is reduced. In addition, since no gap occurs between the outer surface of the measurement site and the second surface of the dielectric layer, no radio wave propagation loss due to such a gap occurs. Therefore, the received signal level is further stabilized, and the biological information can be measured with high precision.

In the biometric antenna device of one embodiment, a relative permittivity of the dielectric layer at a frequency of the radio wave is set within a range of 1 to 5.

Here, the relative permittivity εᵣ=1 corresponds to the relative permittivity of air. The relative permittivity εᵣ=5 corresponds to the lower limit of the relative permittivity of the living body (measurement site) because the relative permittivity of the living body is in the range of about 5 to 40.

In the biometric antenna device of this embodiment, the relative permittivity (εᵣ) of the dielectric layer at the frequency of the radio wave is set within a range of 1 to 5. Therefore, the relative permittivity (εᵣ) of the dielectric layer and the relative permittivity of the measurement site increase in this order. Therefore, power reflection at the interface between the measurement site and the dielectric layer is reduced. As a result, the signal-to-noise ratio of the received signal is increased, and biological information can be measured with high precision.

In the biometric antenna device of one embodiment, a relative permittivity of the dielectric layer at a frequency of the radio wave is gradually increased from the first surface toward the second surface.

In the biometric antenna device of this embodiment, the relative permittivity (εᵣ) of the dielectric layer at the frequency of the radio wave gradually increases from the first surface (the surface on the side along the conductor layer) toward the second surface (the surface on the side abutting on the measurement site in the mounted state). Therefore, power reflection at the interface between the measurement site and the dielectric layer is reduced. As a result, the SN ratio (signal-to-noise ratio) of the received signal is increased, and biological information can be measured with high precision.

In the biometric antenna device of one embodiment, the dielectric layer has a plurality of cavities dispersed inside the dielectric layer, and therefore, an effective relative permittivity as a whole of the dielectric layer is set lower than a relative permittivity of a material itself of the dielectric layer.

In the biometric antenna device of this embodiment, the dielectric layer has a plurality of cavities dispersed inside the dielectric layer. The relative permittivity of the cavity is approximately equal to 1, and is smaller than the relative permittivity of the material itself of the dielectric layer. Thus, the effective relative permittivity as a whole of the dielectric layer is set lower than the relative permittivity of the material itself of the dielectric layer. Therefore, the degree of freedom for setting the effective relative permittivity as a whole of the dielectric layer is increased.

In the biometric antenna device of one embodiment, the dielectric layer includes a specific portion provided in a range corresponding to the facing surface of the conductor layer or the base material, and a strip-shaped layer portion extending in a strip shape beyond a range occupied by the specific portion, and is constituted to stack the specific portion and the strip-shaped layer portion in a thickness direction.

Here, the “thickness direction” means a direction perpendicular to a direction in which the conductor layer or the dielectric layer spreads in a layer shape (referred to as a “surface direction”).

The biometric antenna device of this embodiment can be mounted on the measurement site in a mode where the strip-shaped layer portion of the dielectric layer winds around the measurement site. Thus, this biometric antenna device is stably mounted on the measurement site.

In addition, in particular, when the strip-shaped layer portion is made of a hygroscopic cloth, even if sweat of the living body occurs in the measurement site, the sweat is absorbed by the strip-shaped layer portion (made of a hygroscopic cloth) of the dielectric layer and is prevented from staying between the outer surface of the measurement site and the second surface of the dielectric layer. As a result, the discomfort of the living body (subject) mounting the biometric antenna device is reduced.

It should be noted that the “strip-shaped layer portion” may constitute a part or a whole of the belt mounted to wind around the measurement site.

In the biometric antenna device of one embodiment, the biometric antenna device further comprises a belt mounted to wind around the measurement site,

wherein the belt is mounted with the conductor layer or the base material and the dielectric layer.

The user's (including a subject. The same applies hereinafter.) winding the belt around the measurement site mounts the biometric antenna device of this embodiment on the measurement site. Thus, this biometric antenna device is stably mounted on the measurement site. In the mounted state, a second surface of the dielectric layer on a side opposite to a side of the first surface on a side along the conductor layer abuts on the outer surface of the measurement site. Then, the dielectric layer keeps the distance (distance in the thickness direction) between the outer surface of the measurement site and the conductor layer constant. Therefore, the received signal level is stabilized, and the biological information can be measured with high precision.

In the biometric antenna device of one embodiment, the dielectric layer includes only a portion corresponding to the facing surface of the conductor layer or the base material, of the belt.

In the biometric antenna device of this embodiment, the dielectric layer includes only a portion corresponding to the facing surface of the conductor layer or the base material, of the belt. Therefore, the configuration of the dielectric layer can be simplified.

In another aspect, a pulse wave measurement device of the present disclosure for measuring a pulse wave of a measurement site of a living body, the pulse wave measurement device comprises:

the biometric antenna device;

wherein the second surface of the dielectric layer is configured to abut on an outer surface of the measurement site, and a transmitting and receiving antenna pair including a transmitting antenna and a receiving antenna formed by the conductor layer is configured to correspond to an artery passing through the measurement site in a mounted state in which the belt is mounted to wind around an outer surface of the measurement site,

a transmitting circuit configured to emit a radio wave toward the measurement site via the transmitting antenna;

a receiving circuit configured to receive a radio wave reflected by the measurement site via the receiving antenna; and

a pulse wave detection unit configured to acquire a pulse wave signal representing a pulse wave of an artery passing through the measurement site based on an output of the receiving circuit.

Here, when the conductor layer is divided into a transmitting antenna and a receiving antenna that receives a radio wave from the transmitting antenna in the surface direction perpendicular to the thickness direction of the conductor layer, the “transmitting and receiving antenna pair” refers to the transmitting antenna and the receiving antenna. In addition, when the conductor layer spatially forms one transmitting and receiving shared antenna, all of the “transmitting antenna”, the “receiving antenna”, and the “transmitting and receiving antenna pair” refer to the transmitting and receiving shared antenna.

The user's winding the belt around the outer surface of the measurement site mounts the pulse wave measurement device of the present disclosure on the measurement site. In the mounted state, the second surface of the dielectric layer abuts on the outer surface of the measurement site. Therefore, the conductor layer faces the outer surface of the measurement site, and the dielectric layer keeps a distance between the outer surface of the measurement site and the conductor layer constant. In addition, a transmitting and receiving antenna pair including a transmitting antenna and a receiving antenna formed by the conductor layer corresponds to an artery passing through the measurement site. In this mounted state, the transmitting circuit emits a radio wave toward the measurement site via the transmitting antenna, that is, from the conductor layer through the dielectric layer (or a gap present on the side of the dielectric layer). In addition, the receiving circuit receives a radio wave reflected by the measurement site with the conductor layer via the receiving antenna, that is, through the dielectric layer (or a gap present on the side of the dielectric layer). The pulse wave detection unit acquires a pulse wave signal representing the pulse wave of the artery passing through the measurement site based on the output of the receiving circuit.

Here, in the mounted state, since the dielectric layer keeps a distance between the outer surface of the measurement site and the conductor layer (which forms the transmitting and receiving antenna pair) constant, the received signal level is stabilized. In particular, since the room between the outer surface of the measurement site and the conductor layer can be made, the range (area) of the measurement site irradiated with a radio wave can be expanded. Therefore, even if the mounting position of the conductor layer is more or less deviated from directly above the radial artery, the signal reflected by the radial artery can be stably received. Therefore, the pulse wave detection unit can acquire a pulse wave signal as biological information with high precision.

In another aspect, a blood pressure measurement device of the present disclosure for measuring blood pressure of a measurement site of a living body, the blood pressure measurement device comprises:

the two sets of pulse wave measurement devices;

wherein a belt in the two sets is integrally formed,

wherein transmitting and receiving antenna pairs in the two sets are arranged apart from each other in a width direction of the belt,

wherein in a mounted state where the belt is mounted to wind around an outer surface of the measurement site, the second surface of the dielectric layer abuts on an outer surface of the measurement site, and a first set of transmitting and receiving antenna pair of the two sets corresponds to an upstream side portion of an artery passing through the measurement site, while a second set of transmitting and receiving antenna pair corresponds to a downstream side portion of the artery,

wherein in each of the two sets, the transmitting circuit emits a radio wave toward the measurement site via the transmitting antenna, and the receiving circuit receives a radio wave reflected by the measurement site via the receiving antenna, and

wherein in each of the two sets, the pulse wave detection unit acquires a pulse wave signal representing a pulse wave of an artery passing through the measurement site based on an output of the receiving circuit,

a time difference acquisition unit configured to acquire a time difference between pulse wave signals acquired by the two sets of respective pulse wave detection units as a pulse transit time; and

a first blood pressure calculation unit configured to calculate a blood pressure value based on a pulse transit time acquired by the time difference acquisition unit by using a predetermined correspondence formula between a pulse transit time and a blood pressure.

In the blood pressure measurement device of the present disclosure, the belt in the two sets is integrally formed, and the transmitting and receiving antenna pairs in the two sets are arranged apart from each other in the width direction of the belt. In the mounted state in which the belt is mounted to wind around the outer surface of the measurement site, in each of the two sets, the second surface of the dielectric layer abuts on the outer surface of the measurement site. Therefore, the conductor layer faces the measurement site, and the dielectric layer keeps a distance between the outer surface of the measurement site and the conductor layer constant. In addition, the first set of transmitting and receiving antenna pair of the two sets corresponds to the upstream side portion of the artery passing through the measurement site, while the second set of transmitting and receiving antenna pair corresponds to the downstream side portion of the artery. In this mounted state, in each of the two sets, the transmitting circuit emits a radio wave toward the measurement site via the transmitting antenna, and the receiving circuit receives a radio wave reflected by the measurement site via the receiving antenna. Specifically, in the first set, the transmitting circuit emits a radio wave toward the upstream side portion of the artery via the transmitting antenna, that is, from the conductor layer through the dielectric layer (or a gap present on the side of the dielectric layer). Along with this, the receiving circuit receives a radio wave reflected by the upstream side portion with the conductor layer via the receiving antenna, that is, through the dielectric layer (or a gap present on the side of the dielectric layer). In addition, in the second set, the transmitting circuit emits a radio wave toward the downstream side portion of the artery via the transmitting antenna, that is, from the conductor layer through the dielectric layer (or a gap present on the side of the dielectric layer). Along with this, the receiving circuit receives a radio wave reflected by the downstream side portion with the conductor layer via the receiving antenna, that is, through the dielectric layer (or a gap present on the side of the dielectric layer). Next, in each of the two sets, the pulse wave detection unit acquires a pulse wave signal representing the pulse wave of the artery passing through the measurement site based on the output of the receiving circuit. Specifically, in the first set, the pulse wave detection unit acquires a pulse wave signal representing the pulse wave of the artery passing through the upstream side portion of the artery based on the output of the receiving circuit. In addition, in the second set, the pulse wave detection unit acquires a pulse wave signal representing the pulse wave of the artery passing through the downstream side portion of the artery based on the output of the receiving circuit. Next, the time difference acquisition unit acquires a time difference between the pulse wave signals acquired by the respective two sets of pulse wave detection units as a pulse transit time. Thereafter, the first blood pressure calculation unit calculates a blood pressure value based on the pulse transit time acquired by the time difference acquisition unit by using a predetermined correspondence formula between the pulse transit time and the blood pressure.

Here, in the blood pressure measurement device, in the mounted state, in each of the two sets, the dielectric layer keeps a distance between the outer surface of the measurement site and the conductor layer (which forms the transmitting and receiving antenna pair) constant. Therefore, in each of the two sets, the received signal level is stable and the pulse wave detection unit can acquire a pulse wave signal as biological information with high precision. As a result, the time difference acquisition unit can acquire the pulse transit time with high precision, and thus the first blood pressure calculation unit can calculate (estimate) the blood pressure value with high precision.

In the blood pressure measurement device of one embodiment, the belt is mounted with a fluid bag for pressing the measurement site, further comprising:

a pressure control unit configured to supply air to the fluid bag to control pressure; and

a second blood pressure calculation unit configured to calculate blood pressure by an oscillometric method based on pressure in the fluid bag.

In the blood pressure measurement device of this embodiment, the blood pressure measurement (estimation) based on the pulse transit time and the blood pressure measurement by the oscillometric method can be performed by using a common belt. Therefore, the convenience of the user is enhanced.

In another aspect, an apparatus of the present disclosure comprises:

the biometric antenna device;

the pulse wave measurement device; or

the blood pressure measurement device.

An apparatus of the present disclosure may include the biometric antenna device, the pulse wave measurement device, or the blood pressure measurement device and may include a functional unit for performing another function. According to this apparatus, biological information can be measured with high precision, pulse wave signals as biological information can be acquired with high precision, or the blood pressure value can be calculated (estimated) with high precision. In addition, this apparatus can perform various functions.

In another aspect, a biological information measurement method of the present disclosure for acquiring biological information from a measurement site of a living body by using the biometric antenna device, the biological information measurement method comprises:

causing the second surface of the dielectric layer to abut on an outer surface of the measurement site to mount the biometric antenna device on the measurement site; and

in a mounted state where the dielectric layer keeps a distance between an outer surface of the measurement site and the conductor layer constant, emitting a radio wave from the conductor layer toward the measurement site through the dielectric layer or a gap present on a side of the dielectric layer, and/or receiving a radio wave reflected by the measurement site with the conductor layer through the dielectric layer or a gap present on a side of the dielectric layer.

According to the biological information measurement method of the present disclosure, in the mounted state, the dielectric layer keeps the distance between the outer surface of the measurement site and the conductor layer constant. In addition, due to the dielectric layer interposed between the outer surface of the measurement site and the conductor layer (or the base material), it is less likely to be affected by fluctuations in the dielectric constant of the living body (the relative permittivity of the living body varies in the range of about 5 to 40). In addition, since room between the outer surface of the measurement site and the conductor layer can be made, the range (area) of the measurement site irradiated with a radio wave can be expanded as compared with the case where the conductor layer is in direct contact with the outer surface of the measurement site. As a result, the received signal level is stabilized. Therefore, according to this biometric antenna device, biological information can be measured with high precision.

In another aspect, a pulse wave measurement method of the present disclosure for measuring a pulse wave of a measurement site of a living body by using the pulse wave measurement device, the pulse wave measurement method comprises:

mounting the belt to wind around an outer surface of the measurement site, causing the second surface of the dielectric layer to abut on an outer surface of the measurement site, and causing a transmitting and receiving antenna pair including a transmitting antenna and a receiving antenna formed by the conductor layer to correspond to an artery passing through the measurement site;

in a mounted state in which the dielectric layer keeps a distance between the measurement site and the conductor layer constant, emitting a radio wave toward the measurement site with the transmitting circuit via the transmitting antenna, and receiving a radio wave reflected by the measurement site with the receiving circuit via the receiving antenna; and

acquiring a pulse wave signal representing a pulse wave of an artery passing through the measurement site with the pulse wave detecting unit based on an output of the receiving circuit.

According to the pulse wave measurement method of the present disclosure, in the mounted state, since the dielectric layer keeps a distance between the outer surface of the measurement site and the conductor layer (which forms the transmitting and receiving antenna pair) constant, the received signal level is stabilized. In particular, since the room between the outer surface of the measurement site and the conductor layer can be made, the range (area) of the measurement site irradiated with a radio wave can be expanded. Therefore, even if the mounting position of the conductor layer is more or less deviated from directly above the radial artery, the signal reflected by the radial artery can be stably received. Therefore, the pulse wave signal as biological information can be acquired with high precision.

In another aspect, a blood pressure measurement method of the present disclosure for measuring blood pressure of a measurement site of a living body by using the blood pressure measurement device, the blood pressure measurement method comprises:

mounting the belt to wind around an outer surface of the measurement site, causing the second surface of the dielectric layer to abut on an outer surface of the measurement site, and causing a first set of transmitting and receiving antenna pair of the two sets to correspond to an upstream side portion of an artery passing through the measurement site, while causing a second set of transmitting and receiving antenna pair to correspond to a downstream side portion of the artery;

in a mounted state where the dielectric layer keeps a distance between the measurement site and the conductor layer constant, in each of the two sets, emitting a radio wave toward the measurement site with the transmitting circuit via the transmitting antenna, and receiving a radio wave reflected by the measurement site with the receiving circuit via the receiving antenna;

in each of the two sets, acquiring a pulse wave signal representing a pulse wave of an artery passing through the measurement site with the pulse wave detection unit based on an output of the receiving circuit;

acquiring a time difference between pulse wave signals acquired by the two sets of respective pulse wave detection units with the time difference acquisition unit as a pulse transit time; and

calculating a blood pressure value with the first blood pressure calculation unit based on a pulse transit time acquired by the time difference acquisition unit by using a predetermined correspondence formula between a pulse transit time and a blood pressure.

According to this blood pressure measurement method, in the mounted state, in each of the two sets, the dielectric layer keeps a distance between the outer surface of the measurement site and the conductor layer (which forms the transmitting and receiving antenna pair) constant. Therefore, in each of the two sets, the received signal level is stable and the pulse wave signal as biological information can be acquired with high precision. As a result, the pulse transit time can be acquired with high precision, and therefore, the blood pressure value can be calculated (estimated) with high precision.

The above embodiments are illustrative, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A biometric antenna device for emitting a radio wave toward a measurement site of a living body or for receiving a radio wave from the measurement site, the biometric antenna device comprising:
a conductor layer configured to face the measurement site for emitting and/or receiving the radio wave; and
a dielectric layer mounted along a facing surface facing the measurement site of the conductor layer or of a base material mounting the conductor layer and extending in parallel with the conductor layer, the dielectric layer having a predetermined relative permittivity,
wherein the dielectric layer keeps a distance between an outer surface of the measurement site and the conductor layer constant, in a mounted state in which a second surface on a side opposite to a side of a first surface on a side along the conductor layer of the dielectric layer abuts on an outer surface of the measurement site.

2. The biometric antenna device according to claim 1, wherein the conductor layer or the base material and the dielectric layer have flexibility configured to be deformed along an outer surface of the measurement site as a whole.

3. The biometric antenna device according to claim 1, wherein a relative permittivity of the dielectric layer at a frequency of the radio wave is set within a range of 1 to 5.

4. The biometric antenna device according to claim 1, wherein a relative permittivity of the dielectric layer at a frequency of the radio wave is gradually increased from the first surface toward the second surface.

5. The biometric antenna device according to claim 1, wherein the dielectric layer has a plurality of cavities dispersed inside the dielectric layer, and therefore, an effective relative permittivity as a whole of the dielectric layer is set lower than a relative permittivity of a material itself of the dielectric layer.

6. The biometric antenna device according to claim 1, wherein the dielectric layer includes a specific portion provided in a range corresponding to the facing surface of the conductor layer or the base material, and a strip-shaped layer portion extending in a strip shape beyond a range occupied by the specific portion, and is constituted to stack the specific portion and the strip-shaped layer portion in a thickness direction.

7. The biometric antenna device according to claim 1, further comprising a belt mounted to wind around the measurement site,
wherein the belt is mounted with the conductor layer or the base material and the dielectric layer.

8. The biometric antenna device according to claim 7, wherein the dielectric layer includes only a portion corresponding to the facing surface of the conductor layer or the base material, of the belt.

9. A pulse wave measurement device for measuring a pulse wave of a measurement site of a living body, the pulse wave measurement device comprising:
a biometric antenna device according to claim 7;
wherein the second surface of the dielectric layer is configured to abut on an outer surface of the measurement site, and a transmitting and receiving antenna pair including a transmitting antenna and a receiving antenna formed by the conductor layer is configured to correspond to an artery passing through the measurement site in a mounted state in which the belt is mounted to wind around an outer surface of the measurement site,
a transmitting circuit configured to emit a radio wave toward the measurement site via the transmitting antenna;
a receiving circuit configured to receive a radio wave reflected by the measurement site via the receiving antenna; and
a pulse wave detection unit configured to acquire a pulse wave signal representing a pulse wave of an artery passing through the measurement site based on an output of the receiving circuit.

10. A blood pressure measurement device for measuring blood pressure of a measurement site of a living body, the blood pressure measurement device comprising:
two sets of pulse wave measurement devices according to claim 9;
wherein a belt in the two sets is integrally formed,
wherein transmitting and receiving antenna pairs in the two sets are arranged apart from each other in a width direction of the belt,
wherein in a mounted state where the belt is mounted to wind around an outer surface of the measurement site, the second surface of the dielectric layer abuts on an outer surface of the measurement site, and a first set of transmitting and receiving antenna pair of the two sets corresponds to an upstream side portion of an artery passing through the measurement site, while a second set of transmitting and receiving antenna pair corresponds to a downstream side portion of the artery,
wherein in each of the two sets, the transmitting circuit emits a radio wave toward the measurement site via the transmitting antenna, and the receiving circuit receives a radio wave reflected by the measurement site via the receiving antenna, and
wherein in each of the two sets, the pulse wave detection unit acquires a pulse wave signal representing a pulse wave of an artery passing through the measurement site based on an output of the receiving circuit,
a time difference acquisition unit configured to acquire a time difference between pulse wave signals acquired by the two sets of respective pulse wave detection units as a pulse transit time; and
a first blood pressure calculation unit configured to calculate a blood pressure value based on a pulse transit time acquired by the time difference acquisition unit by using a predetermined correspondence formula between a pulse transit time and a blood pressure.

11. The blood pressure measurement device according to claim 10, wherein the belt is mounted with a fluid bag for pressing the measurement site, further comprising:
a pressure control unit configured to supply air to the fluid bag to control pressure; and
a second blood pressure calculation unit configured to calculate blood pressure by an oscillometric method based on pressure in the fluid bag.

12. An apparatus comprising:
the biometric antenna device according to claim 1.

13. A biological information measurement method for acquiring biological information from a measurement site of a living body by using the biometric antenna device according to claim 1, the biological information measurement method comprising:
causing the second surface of the dielectric layer to abut on an outer surface of the measurement site to mount the biometric antenna device on the measurement site; and
in a mounted state where the dielectric layer keeps a distance between an outer surface of the measurement site and the conductor layer constant, emitting a radio wave from the conductor layer toward the measurement site through the dielectric layer or a gap present on a side of the dielectric layer, and/or receiving a radio wave reflected by the measurement site with the conductor layer through the dielectric layer or a gap present on a side of the dielectric layer.

14. A pulse wave measurement method for measuring a pulse wave of a measurement site of a living body by using the pulse wave measurement device according to claim 10, the pulse wave measurement method comprising:
mounting the belt to wind around an outer surface of the measurement site, causing the second surface of the dielectric layer to abut on an outer surface of the measurement site, and causing a transmitting and receiving antenna pair including a transmitting antenna and a receiving antenna formed by the conductor layer to correspond to an artery passing through the measurement site;
in a mounted state in which the dielectric layer keeps a distance between the measurement site and the conductor layer constant, emitting a radio wave toward the measurement site with the transmitting circuit via the transmitting antenna, and receiving a radio wave reflected by the measurement site with the receiving circuit via the receiving antenna; and
acquiring a pulse wave signal representing a pulse wave of an artery passing through the measurement site with the pulse wave detecting unit based on an output of the receiving circuit.

15. A blood pressure measurement method for measuring blood pressure of a measurement site of a living body by using the blood pressure measurement device according to claim 11, the blood pressure measurement method comprising:
mounting the belt to wind around an outer surface of the measurement site, causing the second surface of the dielectric layer to abut on an outer surface of the measurement site, and causing a first set of transmitting and receiving antenna pair of the two sets to correspond to an upstream side portion of an artery passing through the measurement site, while causing a second set of transmitting and receiving antenna pair to correspond to a downstream side portion of the artery;
in a mounted state where the dielectric layer keeps a distance between the measurement site and the conductor layer constant, in each of the two sets, emitting a radio wave toward the measurement site with the transmitting circuit via the transmitting antenna, and receiving a radio wave reflected by the measurement site with the receiving circuit via the receiving antenna;
in each of the two sets, acquiring a pulse wave signal representing a pulse wave of an artery passing through the measurement site with the pulse wave detection unit based on an output of the receiving circuit;
acquiring a time difference between pulse wave signals acquired by the two sets of respective pulse wave detection units with the time difference acquisition unit as a pulse transit time; and
calculating a blood pressure value with the first blood pressure calculation unit based on a pulse transit time acquired by the time difference acquisition unit by using a predetermined correspondence formula between a pulse transit time and a blood pressure.
